(19) United States
(12) Patent Application Publication
(10) Pub. No.: US 2025/0186746 A1
(43) Pub. Date: Jun. 12, 2025

(54) INTRODUCER WITH EXPANDABLE PLUG

(71) Applicant: EDWARDS LIFESCIENCES CORPORATION, Irvine, CA (US)

(72) Inventor: Roy Shitrit, Zichron Yaakov (IL)

(73) Assignee: EDWARDS LIFESCIENCES CORPORATION, Irvine, CA (US)

(21) Appl. No.: 19/061,819

(22) Filed: Feb. 24, 2025

Related U.S. Application Data
(63) Continuation of application No. PCT/US2023/030406, filed on Aug. 16, 2023.
(60) Provisional application No. 63/402,141, filed on Aug. 30, 2022.

(51) Int. Cl. A61M 25/06; A61M 25/00

(52) U.S. Cl. CPC A61M 25/0662; A61M 25/0021; A61M 25/0045; A61M 25/0068; A61M 2025/0004; A61M 2025/0018; A61M 2025/0024; A61M 2025/006

(57) ABSTRACT

Introducers that can be used in combination with sheaths for introducing a delivery apparatus into a patient's vasculature, and methods for utilizing such introducers, are disclosed herein. As one example, an introducer can include a nosecone, and inner shaft attached to the nosecone, and outer shaft disposed around the inner shaft, and a plug disposed around the inner shaft. The plug is configured to expand from a first diameter in a compacted configuration thereof, to a second diameter in an expanded configuration thereof.

### CROSS-REFERENCE TO RELATED APPLICATIONS

This application is a continuation of PCT Application PCT/US2023/030406, filed Aug. 16, 2023, which claims the benefit of U.S. Provisional Application No. 63/402,141, filed Aug. 30, 2022, each of which is incorporated by reference herein in its entirety.

### FIELD

The present disclosure relates to introducers that can be used in combination with expandable sheaths for introducing a delivery apparatus into a patient's vasculature.

### BACKGROUND

Delivery apparatuses are used to implant prosthetic devices, such as a prosthetic heart valve, at locations inside the body that are not readily accessible by surgery or where access without invasive surgery is desirable. For example, aortic, mitral, tricuspid, and/or pulmonary prosthetic heart valves can be delivered to a treatment site using minimally invasive surgical techniques. The delivery apparatuses may comprise endovascular catheter assemblies.

An introducer sheath may be used to introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss.

### SUMMARY

Conventional methods of accessing a vessel, such as a femoral artery, prior to introducing the delivery apparatus include dilating the vessel using multiple dilators or sheaths that progressively increase in diameter. This repeated insertion and vessel dilation can increase the time the procedure takes, as well as the risk of damage to the vessel. Accordingly, improvements in systems, apparatuses, and methods of introducing a delivery apparatus into a patient's vasculature are desired.

The present disclosure is directed toward introducers of introducer device assemblies, that include an expandable plug configured to expand from a first plug diameter in a compacted configuration of the plug, to a second plug diameter in an expanded configuration of the plug. Upon expansion, the plug can be withdrawn in a proximal direction along a lumen of an expandable sheath, to expand the sheath prior to insertion of a delivery apparatus carrying a prosthetic implant. The sheath accordingly may be expanded without requiring insertion of multiple dilators of sheaths that progressively increase in diameter.

According to one aspect of the disclosure, an introducer device assembly includes an introducer which comprises a nosecone, an inner shaft, an outer shaft disposed around the inner shaft, and a plug disposed around the inner shaft and positioned between the outer shaft and the nosecone, wherein plug is configured to expand from a plug first diameter in a compacted configuration of the plug, to a second plug diameter in an expanded configuration of the plug

In some aspects, the nosecone tapers from a nosecone proximal end to a nosecone distal tip.

In some aspects, the inner shaft comprises an inner shaft distal end attached to the nosecone.

In some aspects, the outer shaft comprises an outer shaft distal end and an outer shaft proximal end, wherein the outer shaft distal end is proximal to the inner shaft distal end.

In some aspects, the plug comprises a plug distal end and a plug proximal end, wherein the plug distal end is affixed to the nosecone and the plug proximal end is affixed to the outer shaft distal end.

In some aspects, the plug comprises a distal portion, a proximal portion, and an intermediate portion, wherein the distal portion extends proximally from the plug distal end, wherein the proximal portion extends distally from the plug proximal end, and wherein the intermediate portion extends between the distal portion and the proximal portion.

In some aspects, approximation of the plug distal end and the plug proximal end to each other is configured to cause the plug to expand from the plug first diameter to the plug second diameter.

In some aspects, the intermediate portion has a uniform diameter equal to the second plug diameter along an intermediate portion length thereof when the plug is in the expanded configuration.

In some aspects, at least one of the inner shaft and the outer shaft is axially movable relative to the other.

In some aspects, the introducer device assembly further comprises a sheath having a sheath distal end and a sheath lumen, wherein the sheath has a natural diameter in a non-expanded state thereof, and is expandable in a direction radially outward from a central axis of the sheath to a diameter which is greater than its natural diameter, and wherein the introducer is configured to move within the sheath lumen.

In some aspects, proximal movement of the plug in the expanded configuration through the sheath lumen is configured to cause the sheath to locally expand in response to the outwardly directed radial force provided by the plug.

In some aspects, the sheath includes at least one polymeric layer that includes a plurality of longitudinally-extending folds when the sheath is in the natural diameter, the plurality of longitudinally-extending folds creating a plurality of circumferentially spaced ridges and a plurality of circumferentially spaced valleys, and wherein proximal movement of the plug in the expanded configuration through the sheath lumen is configured to cause the sheath to expand radially by levelling out the ridges and the valleys.

In some aspects, the intermediate portion length in the expanded configuration is at least as great as half the second plug diameter.

In some aspects, the outer shaft defines an outer diameter, and wherein the intermediate portion length in the expanded configuration is greater than the outer diameter of the outer shaft.

In some aspects, the plug comprises a plurality of struts longitudinally extending between the plug distal end and the plug proximal end.

In some aspects, the plug comprises a plurality of axial support sections and a plurality of circumferential support sections, wherein the plurality of axial support sections are parallel, in the compacted configuration, to a central axis of the plug, and wherein the plurality of circumferential support sections extend around a circumference of the plug.

In some aspects, the plug comprises a plurality of struts that comprises a plurality of angled struts and a plurality of circumferential struts, wherein the plurality of angled struts are angled with respect to a central axis of the plug, and wherein the plurality of circumferential struts extend around a circumference of the plug.

In some aspects, the introducer further comprises at least one stopper arm attached at a stopper proximal end to an outer surface of the inner shaft, and terminating with a stopper distal free end which is biased radially outward, wherein the stopper distal free end is axially positioned distal to the outer shaft proximal end when the plug is in the compacted configuration, and is axially positioned proximal to the outer shaft proximal end when the plug is in the expanded configuration.

In some aspects, the stopper arm is retained in a folded configuration between the inner shaft and the outer shaft when the plug is in the compacted configuration.

In some aspects, the stopper arm is biased radially outward such that the stopper distal free end abuts the outer shaft proximal end when the plug is in the expanded configuration.

In some aspects, the stopper arm is configured to prevent distal movement of the inner shaft relative to the outer shaft, and/or proximal movement of the outer shaft relative to the inner shaft, when the plug is in the expanded configuration.

According to one aspect of the disclosure, a method for expanding a sheath comprises steps of inserting and introducer device assembly that includes the sheath and an introducer into a patient's vasculature, expanding a plug of the introducer, and axially sliding the plug relative to the sheath to expand at least a portion of the sheath in a direction radially outward from a lumen of the sheath.

In some aspects, the sheath extends around the introducer and has a sheath distal end, and is configured to expand in a direction radially outward from a central axis of the sheath.

In some aspects, the introducer comprises a nosecone.

In some aspects, the introducer comprises an inner shaft having an inner shaft distal end attached to the nosecone.

In some aspects, the introducer comprises an outer shaft disposed around the inner shaft and having an outer shaft distal end proximal to the inner shaft distal end.

In some aspects, the plug of the introducer disposed around the inner shaft.

In some aspects, the plug comprises a plug distal end affixed to the nosecone, a plug proximal end affixed to the outer shaft distal end, and an intermediate portion disposed between the plug distal end and the plug proximal end.

In some aspects, the step of expanding the plug comprises approximating the plug distal end and the plug proximal end to each other, thereby expanding the plug from a plug first diameter in a compacted configuration of the plug, to a second plug diameter in an expanded configuration of the plug.

In some aspects, the intermediate portion exhibits a uniform diameter equal to the second plug diameter along an intermediate portion length thereof in the expanded configuration.

In some aspects, the outer shaft defines an outer diameter, wherein the intermediate portion length in the expanded configuration is greater than the outer diameter of the outer shaft.

In some aspects, the intermediate portion length in the expanded configuration is at least as great as half the second plug diameter.

In some aspects, the sheath comprises at least one polymeric layer that includes a plurality of longitudinally-extending folds when the sheath is in a natural unexpanded diameter, wherein the step of sliding the plug in a proximal direction comprises at least partially unfolding the plurality of longitudinally-extending folds.

In some aspects, the step of at least partially unfolding the plurality of longitudinally-extending folds decreases a wall thickness of the sheath.

In some aspects, the sheath comprises at least one polymeric layer that includes a plurality of longitudinally-extending folds when the sheath is in a natural unexpanded diameter, the plurality of longitudinally-extending folds creating a plurality of circumferentially spaced ridges and a plurality of circumferentially spaced valleys, wherein the step of sliding the plug in a proximal direction comprises levelling out the ridges and the valleys.

According to some aspects of the disclosure, there is provided an introducer comprising a nosecone, an inner shaft, an outer shaft disposed around the inner shaft, and a plug disposed around the inner shaft. The nosecone tapers from a nosecone proximal end to a nosecone distal tip. The inner shaft has an inner shaft distal end attached to the nosecone. The outer shaft has an outer shaft distal end proximal to the inner shaft distal end, and an outer shaft proximal end. The plug has a plug distal end affixed to the nosecone, a plug proximal end affixed to the outer shaft, a distal portion extending proximally from the plug distal end, a proximal portion extending distally from the plug proximal end, and an intermediate portion between the distal portion and the proximal portion. The plug is configured to expand, upon approximation of the plug distal end and the plug proximal end to each other, from a plug first diameter in a compacted configuration of the plug, to a second plug diameter in an expanded configuration of the plug. The intermediate portion exhibits a uniform diameter equal to the second plug diameter along an intermediate portion length thereof in the expanded configuration.

According to some aspects of the disclosure, there is provided an introducer device assembly comprising a sheath having a sheath distal end and a sheath lumen, and an introducer configured to axially move within the sheath lumen. The sheath has a natural diameter in a non-expanded state thereof, and is expandable in a direction radially outward from a central axis of the sheath to a diameter which is greater than its natural diameter. The introducer comprises a nosecone, an inner shaft, an outer shaft disposed around the inner shaft, and a plug disposed around the inner shaft. The nosecone tapers from a nosecone proximal end to a nosecone distal tip. The inner shaft has an inner shaft distal end attached to the nosecone. The outer shaft has an outer shaft distal end proximal to the inner shaft distal end, and an outer shaft proximal end. The plug has a plug distal end affixed to the nosecone, a plug proximal end affixed to the outer shaft, a distal portion extending proximally from the plug distal end, a proximal portion extending distally from the plug proximal end, and an intermediate portion between the distal portion and the proximal portion. The plug is configured to expand, upon approximation of the plug distal end and the plug proximal end to each other, from a plug first diameter in a compacted configuration of the plug, to a second plug diameter in an expanded configuration of the plug. The intermediate portion exhibits a uniform diameter equal to the second plug diameter along an intermediate portion length thereof in the expanded configuration.

According to some aspects of the disclosure, there is provided a method comprising a step of inserting an introducer device assembly comprising a sheath and an introducer into a patient's vasculature. The sheath extends around the introducer and has a sheath distal end and a sheath lumen, and is configured to expand in a direction radially outward from a central axis of the sheath. The introducer comprises a nosecone, an inner shaft having an inner shaft distal end attached to the nosecone, an outer shaft disposed around the inner shaft and having an outer shaft distal end proximal to the inner shaft distal end, and a plug disposed around the inner shaft. The plug has a plug distal end affixed to the nosecone, a plug proximal end affixed to the outer shaft distal end, and an intermediate portion disposed between the plug distal end and the plug proximal end. The method further comprises a step of approximating the plug distal end and the plug proximal end to each other to expand the plug from a plug first diameter in a compacted configuration of the plug, to a second plug diameter in an expanded configuration of the plug. The intermediate portion exhibits a uniform diameter equal to the second plug diameter along an intermediate portion length thereof in the expanded configuration. The method further comprises a step of axially sliding the plug relative to the sheath to expand at least a portion of the sheath in a direction radially outward from the sheath lumen.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, which proceeds with reference to the accompanying figures.

### BRIEF DESCRIPTION OF THE FIGURES

Some examples of the disclosed technology are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the disclosed technology. For the sake of clarity, some objects depicted in the figures are not to scale.

In the Figures:

FIG. 1 illustrates an exemplary delivery apparatus for a cardiovascular prosthetic device.

FIG. 2 illustrates an exemplary introducer device assembly.

FIG. 3 illustrates a side view of an expandable sheath that can be used in combination with the introducer device assembly of FIG. 2.

FIG. 4 is a side cross-sectional view of a portion of the expandable sheath of FIG. 3.

FIG. 5 is a magnified view of a portion of the expandable sheath of FIG. 3.

FIG. 6A is a magnified view of a portion of the expandable sheath of FIG. 3 with the outer layer removed for purposes of illustration.

FIG. 6B is a magnified view of a portion of the braided layer of the sheath of FIG. 3.

FIG. 7 is a magnified view of a portion of the expandable sheath of FIG. 3 illustrating expansion of the sheath as a prosthetic device is advanced through the sheath.

FIG. 8A shows a sectional view of the introducer in a compacted configuration of the plug, according to one example.

FIG. 8B shows a sectional view of the introducer of FIG. 8A in an expanded configuration of the plug.

FIG. 9 shows a side view of a distal portion of an introducer device assembly that includes the exemplary expanded plug of FIG. 8B.

FIG. 10A shows a side view of a portion of an exemplary introducer device assembly with an expanded plug concealed within the expandable sheath.

FIG. 10B shows a magnified view of a portion of the introducer device assembly of FIG. 10A.

FIG. 11A illustrates an expanded plug passing along a relatively linear section of the sheath.

FIG. 11B illustrates the expanded plug passing along a relatively curved or bent section of the sheath.

FIGS. 12A and 12B show examples of introducers having expandable plugs with non-identical distal and proximal portions.

FIG. 13A shows a distal portion of an exemplary introducer device assembly with a sheath having a distal end portion defining a tapering tip portion.

FIG. 13B shows a sectional view across line 13B-13B of FIG. 13A.

FIG. 14A shows a sectional view of a portion of the introducer device assembly of FIGS. 8A-8B, with the plug positioned in its compacted state within the sheath lumen.

FIG. 14B shows the plug expanded in the position of FIG. 14A.

FIG. 14C shows a side view of the introducer device assembly of FIG. 14A, with the expanded plug passing through the distal end portion of the sheath to expand it.

FIG. 15A shows a side view of another example of a plug in a compacted configuration.

FIG. 15B shows a side view of the plug FIG. 15A in an expanded configuration.

FIG. 16 shows a sectional view of the expanded plug of FIG. 15B.

FIG. 17A shows a side view of yet another example of a plug in a compacted configuration.

FIG. 17B shows a side view of the plug FIG. 17A in an expanded configuration.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” as used herein refer to regions of a sheath, catheter, or delivery assembly. “Proximal” means that region closest to handle of the device, while “distal” means that region farthest away from the handle of the device. The term “proximal” when used with two spatially separated positions or parts of an object can be understood to mean closer to or oriented towards the proximal end of the device or apparatus. The term “distal” when used with two spatially separated positions or parts of an object can be understood to mean closer to or oriented towards the distal end of the device or apparatus. The terms “longitudinal” and “axial” are interchangeable, and refer to an axis extending in the proximal and distal directions, unless otherwise expressly defined.

It should be understood that the disclosed examples can be adapted to deliver and implant prosthetic devices in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

In order to avoid undue clutter from having too many reference numbers and lead lines on a particular drawing, some components will be introduced via one or more drawings and not explicitly identified in every subsequent drawing that contains that component.

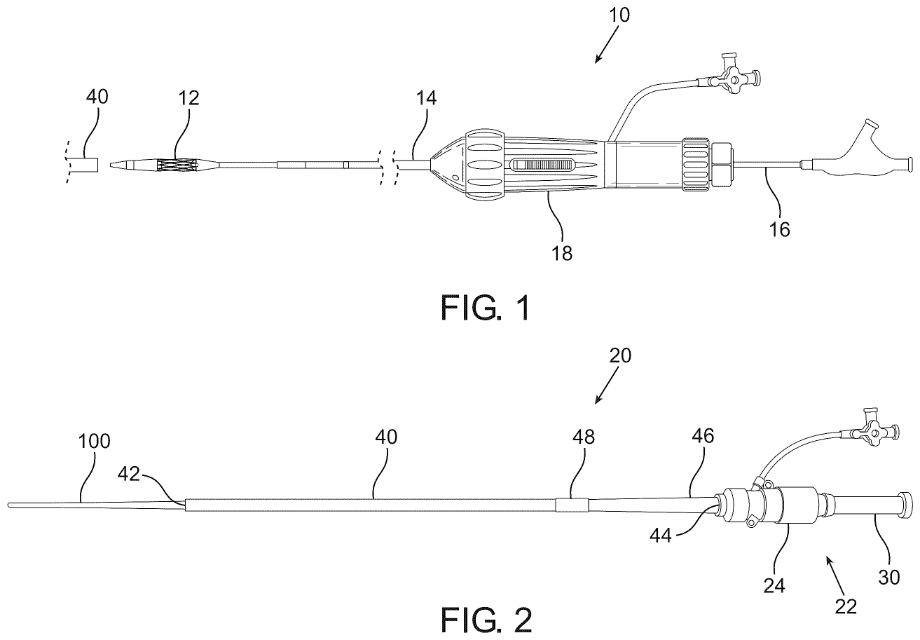

FIG. 1 illustrates an exemplary sheath 40 in use with a representative delivery apparatus 10, such as a prosthetic heart valve or other prosthetic implant, to a patient. The delivery apparatus 10 illustrated can generally include a steerable guide catheter 14 and a balloon catheter 16 extending through the guide catheter 14. A prosthetic device, such as a prosthetic heart valve 12, can be positioned on the distal end of the balloon catheter 16. The guide catheter 14 and the balloon catheter 16 can be adapted to slide longitudinally relative to each other to facilitate delivery and positioning of a prosthetic heart valve 12 at an implantation site in a patient's body. The sheath 40 is an elongate, expandable tube that can include a hemostasis valve at the proximal end of the sheath to stop blood leakage. The guide catheter 14 includes a handle portion 18 and an elongated guide tube or shaft extending from the handle portion 18.

The prosthetic heart valve 12 can be delivered into a patient's body in a radially compressed configuration and radially expanded to a radially expanded configuration at the desired deployment site. In the illustrated example, the prosthetic heart valve 12 is a plastically expandable prosthetic valve that is delivered into the patient's body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Further details regarding a plastically expandable heart valve that can be implanted using the devices disclosed herein are disclosed in U.S. Publication No. 2012/0123529, which is incorporated herein by reference. In other examples, the prosthetic heart valve 12 can be a self-expandable heart valve that is restrained in a radially compressed configuration by a sheath or other component of the delivery apparatus and self-expands to a radially expanded configuration when released by the sheath or other component of the delivery apparatus. Further details regarding a self-expandable heart valve that can be implanted using the devices disclosed herein are disclosed in U.S. Publication No. 2012/0239142, which is incorporated herein by reference. In still other examples, the prosthetic heart valve 12 can be a mechanically expandable heart valve that comprises a plurality of struts connected by hinges or pivot joints and is expandable from a radially compressed configuration to a radially expanded configuration by actuating an expansion mechanism that applies an expansion force to the prosthetic valve.

Further details regarding a mechanically expandable heart valve that can be implanted using the devices disclosed herein are disclosed in U.S. Publication No. 2018/0153689, which is incorporated herein by reference. In still other examples, a prosthetic valve can incorporate two or more of the above-described technologies. For example, a self-expandable heart valve can be used in combination with an expansion device to assist expansion of the prosthetic heart valve.

FIG. 2 illustrates an example of an introducer device assembly 20. The assembly 20 may include the sheath 40 and an introducer 100. The introducer 100 may be positioned within a lumen 50 (indicated in FIG. 4) of the sheath 40, as shown in FIG. 2. A control housing 22 may be positioned at a proximal end of the assembly and may include a sheath housing 24 and an introducer housing 30. The sheath housing 24 and introducer housing 30 may couple together, as shown in FIG. 2.

The sheath 40 and introducer 100 are shown in an insertion configuration, for insertion together into the patient's vasculature. Upon insertion into the patient's vasculature, the introducer 100 may be withdrawn longitudinally from the sheath 40, leaving the sheath 40 within the patient's vasculature. Features of the sheath 40 and the introducer 100 individually are discussed below, as well as the operation of the sheath 40 and introducer 100 together.

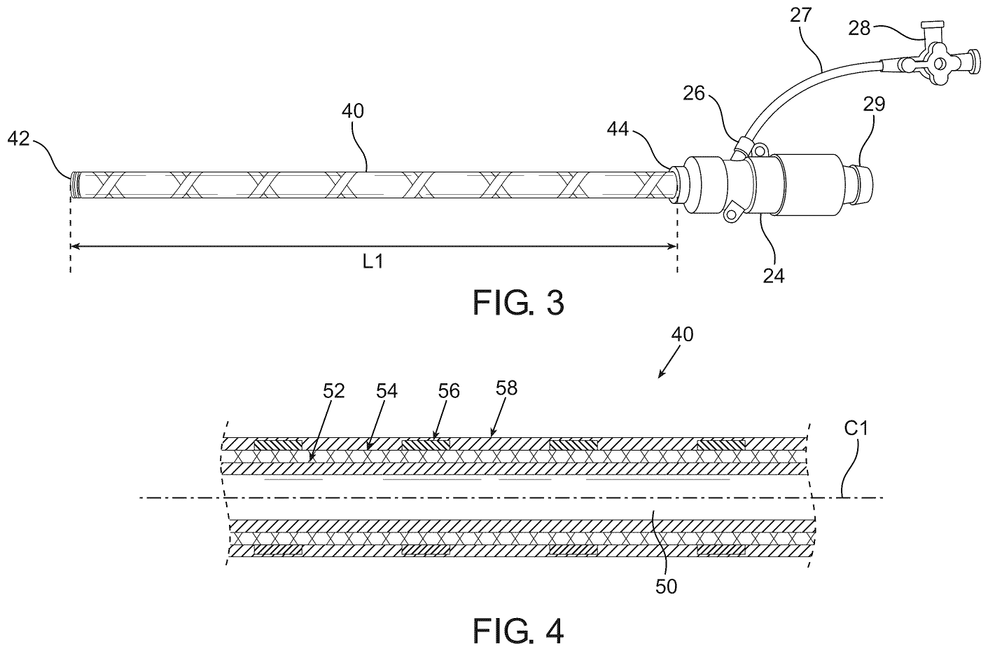

The sheath 40 comprises an elongate body that may have a cylindrical shape. The sheath 40 has a sheath distal end 42 and a sheath proximal end 44, and a length L1 (see FIG. 3) extending from the sheath distal end 42 to the sheath proximal end 44. The sheath 40 is configured to be inserted into a patient's vasculature. The sheath 40 may comprise an introducer sheath that is used to introduce a delivery apparatus into the patient's vasculature.

The vasculature may comprise the blood vessels of the patient's body which may include the femoral artery or other vessels of the patient's body. The vasculature, such as the femoral artery, may be narrow or stiff, and may be difficult to easily insert a delivery apparatus therein. For example, the delivery apparatus may be larger than the vasculature, or may be unwieldy to penetrate through the skin or vasculature of the patient to pass therethrough by itself. Also, the vasculature may be too fragile to receive the delivery apparatus without use of an introducer sheath.

The sheath 40 accordingly may be inserted into the patient's vasculature prior to the delivery apparatus being introduced, to provide an entryway or guide path for the delivery apparatus 10 to introduce the delivery apparatus into the patient's vasculature. After the sheath 40 is inserted, the sheath 40 may remain positioned within and surrounded by the patient's vasculature. The delivery apparatus 10 may then be passed through the lumen of the sheath 40 for introduction into the patient's body. The sheath 40 may remain in the vasculature until a desired time to remove the sheath 40.

The sheath 40 may be inserted into the vasculature percutaneously or a portion of the patient's body may be surgically opened for the sheath 40 to access the vasculature. The delivery apparatus 10 passes through the lumen of the sheath 40 to reach a desired position in the patient's body. As shown in FIG. 1, the delivery apparatus 10 may pass through an opening at the proximal end of the sheath 40 (the control housing 22 is not shown in FIG. 1) for passage through the lumen of the sheath 40 and the vasculature of the patient.

The delivery apparatus and the assemblies disclosed herein may be used in transcatheter aortic valve implantation (TAVI). The delivery apparatus and the systems disclosed herein may be utilized for transarterial access, including transfemoral access, to a patient's heart.

The sheath 40 may include a strain relief portion 46 at the sheath proximal end 44. The strain relief portion 46 may be sized larger than a proximate portion of the sheath 40 and may seal the entry point of the vasculature, to reduce the possibility of blood or other fluid being released between the exterior surface of the sheath 40 and the vasculature. A seal 48 may be positioned along the length of the sheath 40 to further prevent blood or other fluid flow from passing around the sheath 40 toward and out of the sheath proximal end 44.

FIG. 3 illustrates a side view of an exemplary expandable sheath 40 that can be used in the introducer device assembly of FIG. 2. As shown, the sheath housing 24 may be positioned at the sheath proximal end 44. The sheath housing 24 may include an internal chamber (not shown) for the delivery apparatus 10 to be passed through to be delivered to the patient's vasculature. The sheath housing 24 may be configured to remain external to the patient's vasculature when the sheath 40 is inserted therein and may be configured to remain external to the patient's skin for a percutaneous implantation of the sheath 40. The sheath housing 24 may be configured for a user (such as a surgeon) to grip to manipulate the sheath 40.

The sheath housing 24 may comprise a cylindrical body and may include a coupler 29 for coupling to another housing or component of the system. The sheath housing 24 may include a fluid port 26 for passing fluid such as blood to or from the patient's vasculature. Tubing 27 with a valve 28 may be coupled to the fluid port 26, for passing fluid through the fluid port 26 and for sealing flow of the fluid through the fluid port 26.

In some examples, the introducer sheath need not include a sheath housing 24. For example, the sheath 40 can be an integral part of a component of the delivery apparatus 10, such as the guide catheter. For example, the sheath can extend from the handle portion 18 of the guide catheter. Additional examples of introducer devices and expandable sheaths can be found in U.S. Pat. No. 11,273,062, which is incorporated by reference in its entirety.

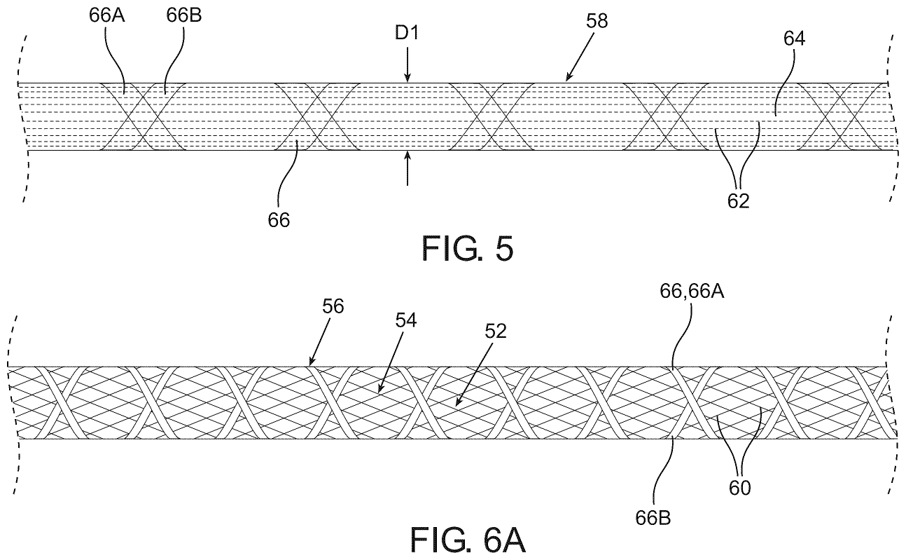

FIGS. 4 and 5 illustrate a cross-sectional view and a side view, respectively, of a portion of the expandable sheath 40. As shown in FIG. 5, the sheath 40 can have a natural, unexpanded outer diameter D1. In certain examples, the expandable sheath 40 can comprise a plurality of co-axial layers extending along at least a portion of the length L1 of the sheath (FIG. 3). For example, with reference to FIG. 4, the expandable sheath 40 can include a first layer 52 (also referred to as an inner layer), a second layer 54 disposed around and radially outward of the first layer 52, a third layer 56 disposed around and radially outward of the second layer 54, and a fourth layer 58 (also referred to as an outer layer) disposed around and radially outward of the third layer 56. In the illustrated configuration, the inner layer 52 can define the sheath lumen 50 extending along a central axis C1.

Referring to FIG. 5, when the sheath 40 is in an unexpanded state, the inner layer 52 and/or the outer layer 58 can form longitudinally-extending folds or creases such that the surface of the sheath comprises a plurality of ridges 62 (also referred to herein as “folds”). The ridges 62 can be circumferentially spaced apart from each other by longitudinally-extending valleys 64. When the sheath expands beyond its natural diameter D1, the ridges 62 and the valleys 64 can level out or be taken up as the surface radially expands and the circumference increases, as further described below. When the sheath collapses back to its natural diameter, the ridges 62 and valleys 64 can reform.

In certain examples, the inner layer 52 and/or the outer layer 58 can comprise a relatively thin layer of polymeric material. For example, in some implementations, the thickness of the inner layer 52 can be from 0.01 mm to 0.5 mm, 0.02 mm to 0.4 mm, or 0.03 mm to 0.25 mm. In certain embodiments, the thickness of the outer layer 58 can be from 0.01 mm to 0.5 mm, 0.02 mm to 0.4 mm, or 0.03 mm to 0.25 mm.

In certain examples, the inner layer 52 and/or the outer layer 58 can comprise a lubricious, low-friction, and/or relatively non-elastic material. In some examples, the inner layer 52 and/or the outer layer 58 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 52, such a low coefficient of friction materials can facilitate passage of the prosthetic device through the sheath lumen 50. Other suitable materials for the inner and outer layers can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Some examples of a sheath 40 can include a lubricious liner on the inner surface of the inner layer 52. Examples of suitable lubricious liners include materials that can further reduce the coefficient of friction of the inner layer 52, such as PTFE, polyethylene, polyvinylidine fluoride, and combinations thereof. Suitable materials for a lubricious liner also include other materials desirably having a coefficient of friction of 0.1 or less.

Additionally, some examples of the sheath 40 can include an exterior hydrophilic coating on the outer surface of the outer layer 58. Such a hydrophilic coating can facilitate insertion of the sheath 40 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (e.g., PTFE, polyethylene, polyvinylidine fluoride), are also suitable for use with the sheath 40. Such hydrophilic coatings may also be included on the inner surface of the inner layer 52 to reduce friction between the sheath and the delivery system, thereby facilitating the use and improving safety. In some examples, a hydrophobic coating, such as Perylene, may be used on the outer surface of the outer layer 58 or the inner surface of the inner layer 52 in order to reduce friction.

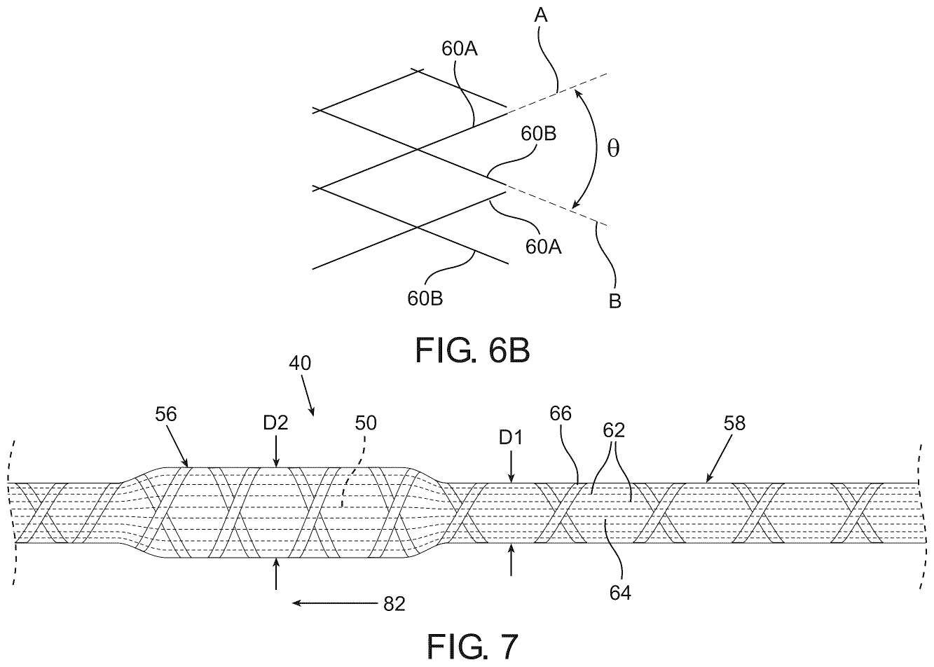

In certain embodiments, the second layer 54 can be a braided layer. FIGS. 6A and 6B illustrate the sheath 40 with the outer layer 58 removed to expose the elastic layer 56. With reference to FIGS. 6A and 6B, the braided layer 54 can comprise a plurality of members or filaments 60 (e.g., metallic or synthetic wires or fibers) braided together. The braided layer 54 can have any desired number of filaments 60, which can be oriented and braided together along any suitable number of axes. For example, with reference to FIG. 6B, the filaments 60 can include a first set of filaments 60A oriented parallel to a first axis A, and a second set of filaments 60B oriented parallel to a second axis B. The filaments 60A and 60B can be braided together in a biaxial braid such that filaments 60A oriented along axis A form an angle θ with the filaments 60B oriented along axis B. In certain examples, the angle θ can be from 5° to 70°, 10° to 60°, 10° to 50°, or 10° to 45°. In the illustrated example, the angle θ is 45°. In other examples, the filaments 60 can also be oriented along three axes and braided in a triaxial braid, or oriented along any number of axes and braided in any suitable braid pattern.

The braided layer 54 can extend along substantially the entire length L1 of the sheath 40, or alternatively, can extend only along a portion of the length of the sheath. In some examples, the filaments 60 can be wires made from metal (e.g., Nitinol, stainless steel, etc.), or any of various polymers or polymer composite materials, such as carbon fiber. In certain examples, the filaments 60 can be round, and can have a diameter of from 0.01 mm to 0.5 mm, 0.03 mm to 0.4 mm, or 0.05 mm to 0.25 mm. In other examples, the filaments 60 can have a flat cross-section with dimensions of 0.01 mm×0.01 mm to 0.5 mm×0.5 mm, or 0.05 mm×0.05 mm to 0.25 mm×0.25 mm. In one embodiment, filaments 60 having a flat cross-section can have dimensions of 0.1 mm×0.2 mm. However, other geometries and sizes are also suitable for certain examples. If a braided wire is used, the braid density can be varied. Some examples have a braid density of from ten picks per inch to eighty picks per inch, and can include eight wires, sixteen wires, or up to fifty-two wires in various braid patterns. In other examples, the second layer 54 can be laser cut from a tube, or laser-cut, stamped, punched, etc., from sheet stock and rolled into a tubular configuration. The layer 54 can also be woven or knitted, as desired.

The third layer 56 can be a resilient, elastic layer (also referred to as an elastic material layer). In certain examples, the elastic layer 56 can be configured to apply force to the underlying layers 52 and 54 in a radial direction (e.g., toward the central axis C1 of the sheath) when the sheath expands beyond its natural diameter by passage of the delivery apparatus through the sheath. Stated differently, the elastic layer 56 can be configured to apply encircling pressure to the layers of the sheath beneath the elastic layer 56 to counteract expansion of the sheath. The radially inwardly directed force is sufficient to cause the sheath to collapse radially back to its unexpanded state after the delivery apparatus is passed through the sheath.

In the illustrated example, the elastic layer 56 can comprise one or more members configured as strands, ribbons, or bands 66 helically wrapped around the braided layer 54. For example, in the illustrated embodiment, the elastic layer 56 comprises two elastic bands 66A and 66B wrapped around the braided layer with opposite helicity, although the elastic layer may comprise any number of bands depending upon the desired characteristics. The elastic bands 66A and 66B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc.

In some examples, the elastic layer can comprise an elastomeric material having a modulus of elasticity of 200 MPa or less. In some examples, the elastic layer 56 can comprise a material exhibiting an elongation to break of 200% or greater, or an elongation to break of 400% or greater. The elastic layer 56 can also take other forms, such as a tubular layer comprising an elastomeric material, a mesh, a shrinkable polymer layer such as a heat-shrink tubing layer, etc. In lieu of, or in addition to, the elastic layer 56, the sheath 40 may also include an elastomeric or heat-shrink tubing layer around the outer layer 58. Examples of such elastomeric layers are disclosed in U.S. Pat. Nos. 9,301,841, 10,792,471, and 10,856,981, which are incorporated herein by reference. In other examples, the elastic layer 56 can also be radially outward of the polymeric layer 58.

In certain examples, one or both of the inner layer 52 and/or the outer layer 58 can be configured to resist axial elongation of the sheath 40 when the sheath expands. For example, one or both of the inner layer 52 and/or the outer layer 58 can resist stretching against longitudinal forces caused by friction between a prosthetic device and the inner surface of the sheath such that the length L1 remains substantially constant as the sheath expands and contracts. As used herein with reference to the length L1 of the sheath, the term “substantially constant” means that the length L1 of the sheath increases by not more than 1%, by not more than 5%, by not more than 10%, by not more than 15%, or by not more than 20%. Meanwhile, with reference to FIG. 6B, the filaments 60A and 60B of the braided layer can be allowed to move angularly relative to each other such that the angle θ changes as the sheath expands and contracts. This, in combination with the longitudinal folds 62 in the layers 52 and 58, can allow the sheath lumen 50 to expand as a prosthetic device is advanced through it.

For example, in some implementations, the inner layer 52 and the outer layer 58 can be heat-bonded during the manufacturing process such that the braided layer 54 and the elastic layer 56 are encapsulated between the layers 52 and 58. More specifically, in certain embodiments, the inner layer 52 and the outer layer 58 can be adhered to each other through the spaces between the filaments 60 of the braided layer 54 and/or the spaces between the elastic bands 66. The layers 52 and 58 can also be bonded or adhered together at the proximal and/or distal ends of the sheath. In certain examples, the layers 52 and 58 are not adhered to the filaments 60. This can allow the filaments 60 to move angularly relative to each other, and relative to the layers 52 and 58, allowing the diameter of the braided layer 54, and thereby the diameter of the sheath, to increase or decrease. As the angle θ between the filaments 60A and 60B changes, the length of the braided layer 54 can also change. For example, as the angle θ increases, the braided layer 54 can foreshorten, and as the angle θ decreases, the braided layer 54 can lengthen to the extent permitted by the areas where the layers 52 and 58 are bonded. However, because the braided layer 54 is not adhered to the layers 52 and 58, the change in length of the braided layer that accompanies a change in the angle θ between the filaments 60A and 60B does not result in a significant change in the length L1 of the sheath.

FIG. 7 illustrates radial expansion of the sheath 40 as a prosthetic device 12 is passed through the sheath in the direction of arrow 82 (e.g., distally). As the prosthetic device 12 is advanced through the sheath 40, the sheath can resiliently expand to a second diameter D2 that corresponds to a size or diameter of the prosthetic device. As the prosthetic device 12 is advanced through the sheath 40, the prosthetic device can apply longitudinal force to the sheath in the direction of motion by virtue of the frictional contact between the prosthetic device and the inner surface of the sheath. However, as noted above, the inner layer 52 and/or the outer layer 58 can resist axial elongation such that the length L1 of the sheath remains constant, or substantially constant. This can reduce or prevent the braided layer 54 from lengthening, and thereby constricting the sheath lumen 50.

Meanwhile, the angle θ between the filaments 60A and 60B can increase as the sheath expands to the second diameter D2 to accommodate the prosthetic valve. This can cause the braided layer 54 to foreshorten. However, because the filaments 60 are not engaged or adhered to the layers 52 or 58, the shortening of the braided layer 54 attendant to an increase in the angle θ does not affect the overall length L1 of the sheath. Moreover, because of the longitudinally-extending folds 62 formed in the layers 52 and 58, the layers 52 and 58 can expand to the second diameter D2 without rupturing, in spite of being relatively thin and relatively non-elastic. In this manner, the sheath 40 can resiliently expand from its natural diameter D1 to a second diameter D2 that is larger than the diameter D1 as a prosthetic device is advanced through the sheath, without lengthening, and without constricting. Thus, the force required to push the prosthetic implant through the sheath is significantly reduced.

Additionally, because of the radial force applied by the elastic layer 56, the radial expansion of the sheath 40 can be localized to the specific portion of the sheath occupied by the prosthetic device. For example, with reference to FIG. 7, as the prosthetic device 12 moves distally through the sheath 40, the portion of the sheath immediately proximal to the prosthetic device 12 can radially collapse back to the initial diameter D1 under the influence of the elastic layer 56. The layers 52 and 58 can also buckle as the circumference of the sheath is reduced, causing the ridges 62 and the valleys 64 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

In addition to the advantages above, the expandable sheath examples described herein can provide surprisingly superior performance relative to known introducer sheaths. For example, it is possible to use a sheath configured as described herein to deliver a prosthetic device having a diameter that is two times larger, 2.5 times larger, or even three times larger than the natural outer diameter of the sheath. For instance, in one example, a crimped prosthetic heart valve having a diameter of 7.2 mm was successfully advanced through a sheath configured as described above and having a natural outer diameter of 3.7 mm. As the prosthetic valve was advanced through the sheath, the outer diameter of the portion of the sheath occupied by the prosthetic valve increased to 8 mm. In other words, it was possible to advance a prosthetic device having a diameter more than two times the outer diameter of the sheath through the sheath, during which the outer diameter of the sheath resiliently increased by 216%. In another example, a sheath with an initial or natural outer diameter of 4.5 mm to 5 mm can be configured to expand to an outer diameter of 8 mm to 9 mm.

In some examples, the sheath 40 may optionally include the layer 52 without the layer 58, or the layer 58 without the layer 52, depending upon the particular characteristics desired.

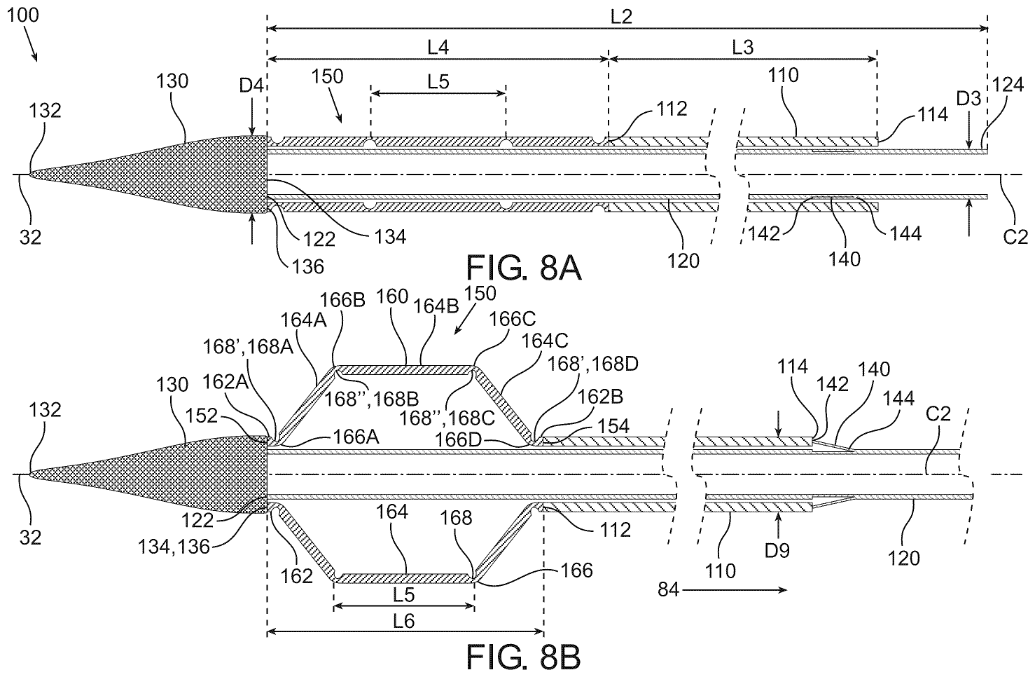

Referring to FIGS. 8A-8B, an introducer 100 may be utilized to introduce sheath 40 into the vasculature of the patient's body. The introducer 100 is configured to be positioned within the sheath lumen 50 to introduce the sheath 40 into the vasculature of the patient's body, as shown in FIG. 2.

FIG. 8A illustrates a cross-sectional side view of the introducer 100 in a compacted configuration. The introducer 100 can include an inner shaft 120 having an inner shaft distal end 122 and an inner shaft proximal end 124, and defining a introducer lumen 106 therein, through which a guidewire 32 (an exposed distal end of which is shown for example in FIGS. 8A-8B) can extend. While not illustrated, the nosecone 130 also include a guidewire lumen, continuous with the introducer lumen 106. The inner shaft 120 can have a length L2 extending from the inner shaft distal end 122 to the inner shaft proximal end 124. The inner shaft 120 of the introducer 100 can be configured to have a length L2 that is greater than the length L1 of the sheath 40, such that a portion of the introducer 100 (e.g., a portion of the inner shaft 120) extends beyond the sheath distal end 42 (marked in FIG. 2).

The introducer 100 may include a nosecone 130 that can be tapered down from a nosecone proximal end 134 to a nosecone distal tip 132. The inner shaft 120 can be attached at its distal end 122 to the nosecone proximal end 134. The nosecone 130 can be configured to extend beyond the sheath distal end 42, as shown in FIG. 2. The tapered nosecone 130 is adapted for insertion into a body tissue.

The introducer can further include an outer shaft 110 disposed over the inner shaft 120. The outer shaft 110 can have an outer shaft distal end 112, an outer shaft proximal end 114, and define a length L3 extending from the outer shaft distal end 112 to the outer shaft proximal end 114. The outer shaft 110 is configured to extend around at least a portion of the inner shaft 120. In some examples, the length L3 of the outer shaft 110 is shorter than the length L2 of the inner shaft 120.

The introducer 100 can further include an expandable plug 150, movable between a compacted configuration, shown in FIG. 8A, and an expanded configuration, shown in FIG. 8B. The expandable plug 150 has a plug distal end 152 and a plug proximal end 154, and defines a plug compacted length L4 extending from the plug distal end 152 to the plug proximal end 154 in a compacted configuration thereof. The plug 150 can be attached at its distal end 152, to the nosecone proximal end 134. The plug can be further attached at its proximal end 154 to the outer shaft distal end 112. Thus, as shown, the plug 150 is disposed around a distal portion of the inner shaft 120, and extends between the nosecone 130 and the outer shaft 110. It is to be understood that the nosecone 130 is shown as a relatively short nosecone in FIGS. 8A-8B for ease of illustration, and that a nosecone of an introducer 100 can be significantly longer in shape (for example, somewhat similar to the shape shown in FIG. 2), and can be relatively flexible, or at least more flexible when compared to a conventional nosecone of a delivery apparatus 10 of the type shown in FIG. 1, to allow it to bend when advanced through tortuous sections of the patient's vasculature without posing a risk of guidewire buckling.

The outer shaft 110 defines an outer diameter D9, the inner shaft 120 defines an outer diameter D3 which is less than D9, and the nosecone 130 defines an outer diameter D4 at its proximal end 144, which can be, in some examples, substantially equal to diameter D9. In some examples, the outer diameter D4 of the nosecone proximal end 134 is greater than the outer diameter D3 of the inner shaft 120, resulting in a step portion 136 of the nosecone proximal end 134 extending radially away from the inner shaft distal end 122, to which the plug distal end 152 can be attached, such as by gluing, welding, and the like. Nevertheless, it is to be understood that any other suitable means of attachment can be employed. For example, the plug can be threadedly engaged with the nosecone.

The term “radially away”, as used with respect to any component of the introducer 100, refers to a direction extending from a central axis C2 passing through the introducer 100 (such as through the introducer lumen 106) and the plug 150, which can be coaxial with the central axis C1 when the introducer 100 extends through the sheath 40. The plug 150 can define an inner side 158 facing the central axis C2, and an opposite outer side 156 facing away from the central axis C2. In some examples, the outer side 156 can include a hydrophilic coating to reduce friction between the sheath 40 and the plug 150. In some examples, the hydrophilic coating includes a material with a low coefficient of friction.

The expandable plug 150 includes a plurality of longitudinally-extending, circumferentially-spaced struts 160, extending between, and optionally terminating at, the plug distal end 152 and the plug proximal end 154. Struts 160 can be formed of a variety of materials and in a variety of shapes or structures sufficient to provide enough strength to cause local expansion of the sheath 40, as will be described in more detail below. For example, each strut 160 can be formed of a stiff polymeric or metallic material. In addition, the expandable plug 150 can include any variety of number of struts 160, so long as the struts are of sufficient number, strength, and/or shape so as to provide sufficient force to an inner surface of the sheath 40 (e.g., an inner surface of inner layer 52) to support local expansion of the sheath 40 as described herein.

Each of the struts 160 further includes at least three strut sections 164, separated by notches 168 configured to form bending points 166 of the struts. Each strut 160 can also include strut end portions 162 extending toward the distal 152 and proximal 154 ends of the plug 150, also separated from adjacent corresponding strut sections 164 by notches 168 configured to form bending points 166.

The plug 150 is configured to transition from a compacted configuration, in which the plug 150 has a first plug diameter, to an expanded configuration, in which the plug 150 has a second plug diameter that is greater than the first plug diameter. The plug 150 is shown in FIG. 8A in a compacted or un-expanded configuration, in which the plug 150 is flattened. In this configuration, all of the strut sections 164 can be substantially parallel to the central axis C2 of the plug. The plug 150 may transition to an expanded or deployed configuration, as shown in FIG. 8B, in which it has a plug second diameter D5 (marked in FIG. 9), which is greater than the plug first diameter, and greater than the natural diameter D1 of the sheath 40. At least one strut section 164 of each strut 160 is spaced away from the inner shaft 120 in this configuration, to a greater extent than in the compacted configuration shown in FIG. 8A.

The plug 150 in the compacted configuration may be configured to fit within the sheath lumen 50. The plug 150 may have a plug first diameter that is sized to fit within the sheath lumen 50. The first plug diameter can be, in some examples, substantially equal to or less than the diameter D4 of the nosecone proximal end 144 (which can be, in some examples, equal to the outer diameter D9 of the outer shaft 110). The first plug diameter can be, in some examples, substantially equal to or less than the diameter D9 of the outer shaft 110. Thus, the plug 150 in the compacted configuration may fit within the sheath lumen 50 without increasing the size of the lumen 50 or otherwise expanding the sheath 40. Any of the diameter D4 of the nosecone proximal end 144, and/or the diameter D9 of outer shaft 110, may be configured to be at or less than the interior diameter of the sheath 40.

In some examples, notches 168 are formed on one side of the struts, which can be either the outer side 156 or the inner side 158. Notching the appropriate bending points 166 facilitates the bending of struts 160 to a desired expanded shape. In the example illustrated in FIGS. 8A-9, each strut 160 includes four notches 168 at bending points 166 disposed between corresponding strut sections 164 and strut end portions 162. Specifically, each strut 160 is shown to include a first strut end portion 162A from the plug distal end 152, a first notch 168A disposed between the first strut end portion 162A and a first strut section 164A, a second notch 168B disposed between the first strut section 164A and a second strut section 164B, a third notch 168C disposed between the second strut section 164B and a third strut section 164C, and a fourth notch 168D disposed between the third strut section 164C and a second strut end portion 162B that terminates at the plug proximal end 154. The first strut end portions 162A can be affixed (e.g., glued, welded, fastened by the use of suitable fasteners, and the like) to the nosecone 130, such as to the nosecone proximal end 134, and the second strut end portions 162B can be similarly affixed to the outer shaft 110, such as to the outer shaft distal end 112. In such cases, the strut end portions 162 can remain parallel to the main axis C2 in both compacted and expanded configurations of the plug 150.

The various notches 168 can include notches 168′ formed at the outer side 156 and notches 168″ formed at the inner side 158, to facilitate a desired direction of bending at the corresponding bending points 166. In the illustrated example, the notches disposed between strut end portions 162 and strut sections 164 are notches 168′ formed at the outer side 156, and those disposed between two subsequent strut sections 164 are notches 168″ formed at the inner side 158. Specifically, a first bending point 166A and fourth bending point 166D, which include notches 168′ formed at the outer side 156, allow the first strut section 164A and third strut section 164C to bend radially outward, away from the respective first strut end portion 162A and second strut end portion 162B. Similarly, second bending point 166B and third bending point 166C, disposed on both sides of the second strut section 164B, allow it to remain relatively parallel with central axis C2, while strut sections 164A and 164C on both sides thereof assume an angled configuration relative to the axis C2 in the expanded configuration. This eventually allows radial displacement of the second strut sections 164B away from axis C2, while remaining substantially parallel with respect to the axis C2.

The plug 150 can be expanded via axial movement of the inner shaft 120 and outer shaft 110 relative to each other, in a manner that approximates the plug distal end 152 and plug proximal end 154, from a distance or plug length L4 in the collapse configuration (see FIG. 8A), to a shorter distance or plug length L6 in the expanded configuration (see FIG. 8B). Since the plug 150 is affixed on both ends thereof to the nosecone 130 and outer shaft 110 in the illustrated example, this is equivalent to approximation of the nosecone proximal end 134 and the outer shaft distal end 112 to each other. In one example, this can be accomplished by pulling the inner shaft 120 in a proximal direction 84 (see FIG. 8B) relative to the outer shaft 110, thereby approximating the nosecone proximal end 134 to the outer shaft distal end 112. In another example, this can be accomplished by pushing the outer shaft 110 in a distal direction 82 relative to the inner shaft 120, thereby approximating the outer shaft distal end 112 to the nosecone proximal end 134. In yet another example, the inner shaft 120 and outer shaft 110 can be simultaneously pulled and pushed, respectively, relative to each other.

The plug 150 can define a distal portion 170 comprising the first strut sections 164A, an intermediate portion 172 comprising the second strut sections 164B, and a proximal portion 174 comprising the third strut sections 164C. The distal and proximal portions 170 and 174 can transition from a relatively cylindrical shape in the compacted configuration of FIG. 8A, to tapering shapes in the expanded configuration extending from a wider diameter D5 to the narrower respective diameters D4 and D9 as shown in FIGS. 8B and 9, such that the projected length of these portions 170, 174 over central axis C2, for example, shortens when transitioning from the compacted to the expanded configuration. In contrast, the projected length L5 of intermediate portion 172, which is the length of second strut section 164B in the illustrated example, remains constant during both the compacted and the expanded configuration of the plug 150. This ensures that in the expanded configuration, the intermediate portion 172 of the plug 150 maintains a length L5, also referred to herein as the intermediate portion length L5, along which it exhibits a uniform diameter, which is the second plug diameter D5. It is to be understood that the intermediate portion length L5 of any exemplary intermediate portion described herein, refers to a length having a non-zero value, such as at least 1 millimeter.

As shown, the expandable plug 150 foreshortens during expansion, such that the length (transitioning between L4 and L6) dictates the diameter of the plug (between any of D4 and D9, and the expanded diameter D5). Thus, approximating the plug distal end 152 and the plug proximal end 154 to a specific distance L6 will result in a specific desired expanded diameter D5. For that end, in some examples, the introducer 100 can include a stopping mechanism configured to prevent re-compression of the plug 150 once expanded to a target pre-set second diameter D5.

In some examples, the introducer 100 can further include one or more stopper arms 140 attached to the outer surface of the inner shaft 120, naturally outwardly biased away from the central axis C2 and from the outer surface of the inner shaft 120. Each stopper arm 140 can extend from a stopper proximal end 144, at which it is coupled to the inner shaft 120, to a stopper distal free end 142. While stopper arms 140 are described herein in the plural form, it is to be understood that any number of stopper arms 140, including a single stopper arm 140, is contemplated.

The stopper arms 140 can be attached to a proximal portion of the inner shaft 120 such that in a compacted configuration of the plug 150, the stopper arms 140 are distal to the outer shaft proximal end 114, during which the arms 140 are folded over the inner shaft 120 and are pressed against the inner surface of the outer shaft 110 as shown in FIG. 8A. The length L2 of the inner shaft 120 can be equal to or greater than the combined length L3 of the outer shaft 110 and the length L4 of the plug 150 in its compacted configuration. Optionally, the inner shaft 120 can include recesses configured to accommodate the stopper arms 140 in their folded state.

As the inner shaft 120 is pulled relative to the outer shaft 110 during plug expansion (or alternatively, as the outer shaft 110 is pushed relative to the inner shaft 120), the arms are axially displaced relative to the outer shaft 110, such that they are fully exposed and can spring radially outward when the plug 150 is expanded to the pre-set second diameter D5. In this position, the stopper distal free ends 142 are both radially and axially aligned with the outer shaft proximal end 114, pressing there-against in a manner that prevents re-compression of the plug 150. This ensures that as soon as the plug 150 reaches the pre-set second diameter D5, it will not be compressed to a narrower diameter.

The length L6 of the plug 150 in its expanded configuration is less than the length L1 of the sheath 40, such that as the plug 150 is slid proximally through the sheath lumen 50, a transient expansion of the sheath 40 occurs. Thus, only a portion of the sheath 40 has a force applied to it and has a localized expansion caused by the plug 150 at a given moment. In some examples, the plug 150 can be advance beyond the sheath distal end 42 prior to expansion thereof, such that that plug 150 can be expanded out of (i.e., distal to) the sheath distal end 42, as shown in FIG. 9 for example. In such cases, pulling the introducer 100 through the sheath lumen 50 in the proximal direction 84 causes the plug 150 to enter through the opening at the sheath distal end 42 and slide within the sheath lumen 50 in the proximal direction 84 to transiently expand the sheath 40 in the direction radially outward from the central axis C1 of the sheath. The transient force applied to a portion of the sheath 40 allows for a lesser force to be applied to the sheath 40 than if the entirety of the sheath 40 were expanded at once. In addition, the sliding motion of the plug 150 reduces static friction that may exist between the plug 150 and the interior surface of the sheath 40. The sliding motion also reduces the possibility of the plug 150 becoming stuck in the lumen 50, due to the kinetic energy provided to the plug 150 and the reduction of adhesive forces that may exist between the outer side 156 of the plug 150 and the inner surface of the sheath 40 if the plug 150 were deployed within the lumen 50.

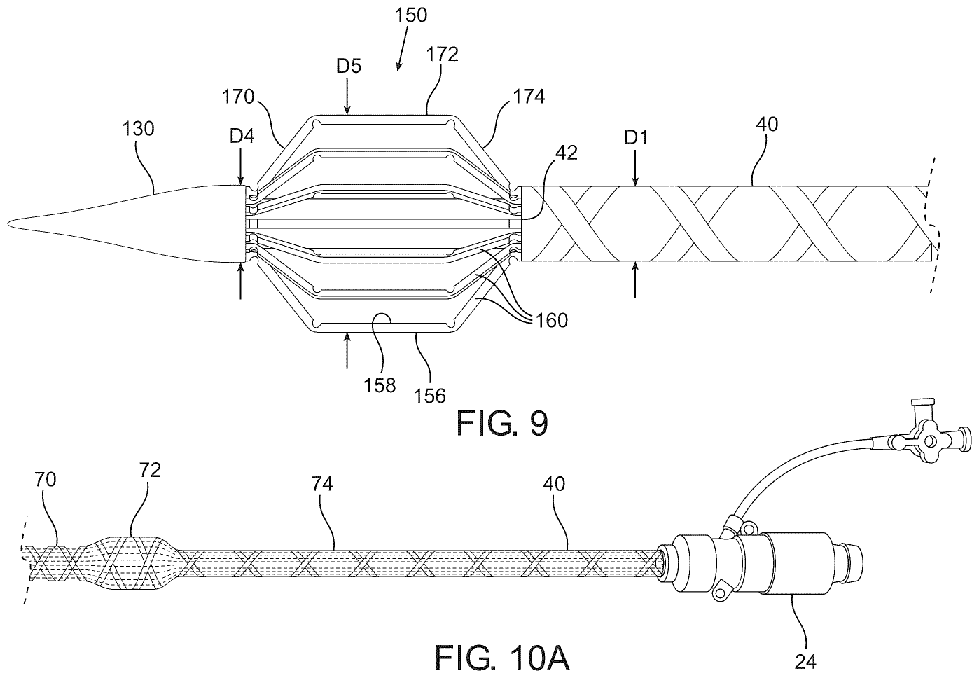

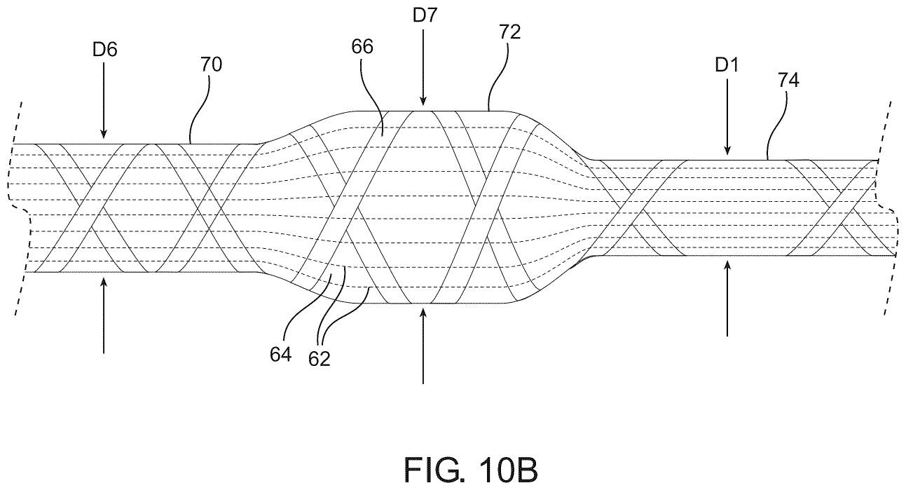

FIG. 10A shows the transient expansion of the sheath 40. The plug 150 is being slid proximally relative to the sheath 40. The location of the plug 150 within the sheath is marked in FIG. 10A as reference number 72. The plug 150 may expand the sheath 40 at the position of the plug 150 and may leave an increased size in the sheath 40 in the distal portion of the sheath 40. FIG. 10B shows a representation of the movement of the plug 150 within the sheath 40. In FIG. 10B, the plug 150 has not yet passed through the proximal portion of the sheath 40 marked as portion 74. The plug 150 is within the portion of the sheath 40 marked as portion 72. The plug 150 has passed through the portion of the sheath 40 marked as portion 70.

The diameter D7 of the sheath 40 at portion 72 is larger than diameter D1 in portion 74 because the plug 150 has not yet passed through portion 74. An outer diameter D7 of the sheath 40 can be equal to the second or expanded diameter D5 plus twice the thickness of the sheath 40. The diameter D6 of the sheath 40 at portion 70 may be less than the diameter D7 at portion 72 because the sheath may be elastic and biased in a direction radially towards the central axis C1. The plug 150 accordingly may be configured to transiently expand a distal portion of the sheath 40, and then an intermediate portion of the sheath 40, and then a proximal portion of the sheath 40 in successive order, rather than expanding a distal, central, and proximal portion of the sheath 40 all at one time. The plug 150 may be sized or have an intermediate portion length L5 of an intermediate portion 172 thereof, relative to the length L1 of the sheath 40, to only expand a portion of the sheath 40 at one time.

The sheath 40 may be configured to partially return to a pre-expanded configuration, as discussed previously. The difference in diameter between portion 70 and portion 74 (i.e., D6-D1), however, may decrease the insertion push force of a delivery apparatus inserted through the sheath 40 after the introducer 100 with plug 150 have been removed from the sheath 40.

The introducer 100 with plug 150 may continue to be withdrawn from the sheath 40 until completely removed from the sheath lumen 50. Upon their removal, a delivery apparatus such as the apparatus 10 shown in FIG. 1 may be inserted into the sheath 40 in a distal direction with a reduced pushing force due to the expanded lumen size.

The system allows for insertion of the sheath 40 and the introducer 100 into the patient's vasculature with a relatively low-profile, or narrow outer diameter. As the introducer 100 is removed from the sheath 40 to leave the sheath 40 positioned within the patient's vasculature, the plug 150 serves to expand the sheath 40, providing a larger lumen 50 size and larger interior diameter. The sheath 40 may partially return to a pre-expanded configuration upon the plug passing therethrough (as marked at portion 70 in FIG. 10B), however, the enhanced size of the sheath 40 nonetheless reduces insertion push force required for the delivery apparatus to be inserted through the sheath 40. The sheath 40 accordingly may be preconditioned for introduction of the delivery apparatus via a plug 150 integrated with the introducer 100.

The system provides benefits over prior methods of sheath expansion. In prior methods, once the introducer (without a plug) is removed from the sheath, one or more dilator rods may be inserted through the sheath in the distal direction to progressively expand the size of the sheath. These prior methods may include multiple steps (e.g., insert sheath and introducer, remove introducer, insert and remove multiple dilator rods) before insertion of the delivery apparatus. The system may alleviate these issues by allowing removal of the introducer with the expanded plug to expand the sheath, without multiple dilator rods needing to be inserted into the sheath. The system may thus provide a reduced number of steps during a medical procedure. In other prior methods, the delivery apparatus is simply pushed through the sheath without the sheath being pre-expanded by a dilator rod or otherwise.

The method can further include removing the introducer with the plug from the expendable sheath such that the outer diameter (e.g., D6) of the sheath (and inner diameter of the sheath lumen) corresponding to the locations along which the plug has been retracted, is greater than the non-expanded, initial diameter of the sheath (e.g., D1).

Lastly, the method can further include inserting a prosthetic implant into the sheath lumen of the expanded sheath, such as prosthetic implant 12 carried by delivery apparatus 10. Advantageously, pre-expansion of the sheath 40 by expandable plug 150 allows the prosthetic implant 12 to be advanced through the sheath 40 at a push force which is less than the push force required for advancement through an un-expanded sheath.

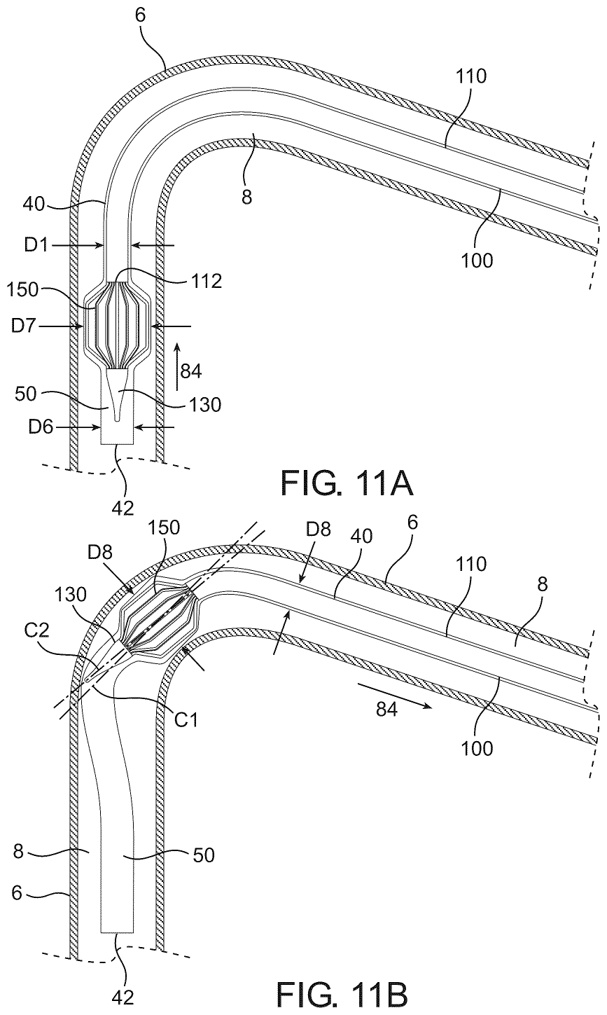

FIGS. 11A-11B illustrate stages in a method of utilizing an introducer device assembly disclosed herein. The method can include insertion of a guidewire 32 (not shown in FIGS. 11A-11B) into a patient's vasculature, such as by accessing the patient's vasculature and passing the guide wire therein until reaching the desired position within the patient's body. This can then be followed by insertion of an introducer and a sheath into the patient's vasculature over the guide wire. The introducer and sheath may be provided according to any of the exemplary introducers and sheaths disclosed herein. The sheath may be configured to expand in a direction radially outward from its central axis C1 as described above. The introducer may be positioned within the sheath lumen upon introduction. The introducer can include an expendable plug retained in a compacted or unexpanded configuration within the sheath lumen upon insertion into the patient's vasculature. In this compacted configuration, the plug can be then advanced to extend at least partially, or completely, out of the sheath lumen (i.e., positioned distal to the sheath distal end) prior to expansion thereof.

The plug can be then expanded from a first diameter in the compacted configuration to a second diameter in the expanded configuration, which is greater than the interior diameter of the sheath lumen. Expansion of the plug can include approximation of the plug distal end and the plug proximal end to each other. Expansion of the plug can include approximation of the nosecone and the outer shaft to each other, and more specifically, approximation of the nosecone proximal end and the outer shaft distal end to each other. Expansion of the plug can include proximally pulling the inner shaft relative to the outer shaft, and/or distally pushing the outer shaft relative to the inner shaft. The plug can include an intermediate portion axially extending along an intermediate portion length L5, parallel to the central axis C2 of the introducer (which is also the central axis of the plug). The plug can then be proximally pulled, in its expanded configuration, through the sheath lumen, toward the sheath proximal end, to expand the sheath in the direction radially outward from its central axis C1.

FIG. 11A illustrates an expendable sheath 40 extending through a lumen 8 of a blood vessel 6. The patient's vasculature may often be tortuous and include regions along which the sheath 40 can follow bent paths along the blood vessel's lumen 8. Both the sheath 40 and the inner and outer shafts 110, 120 of the introducer 100 are made of flexible materials to allow them to pass through curved portions of the vasculature. The plug 150 is shown in FIG. 11A to be positioned within the sheath lumen 50, at a relatively straight or unbent portion of the blood vessel 6 and the sheath 40. In such regions, the plug 150 expands the sheath 40 to a diameter D7 which is greater than the natural or unexpanded diameter D1 of the sheath along the proximal portion of the sheath 74, and wherein the diameter D6 of the distal portion of the sheath 70 can be less than the diameter D7 at the portion in which the plug 150 is positioned (yet also greater than the natural diameter D1), as described above with respect to FIGS. 10A-10B.

FIG. 11B shows the expanded plug 150, after being pulled further in the proximal direction 84, positioned at a bent region of the blood vessel 6 and the sheath 40. In such cases, depending on the extent to which the sheath 40 and/or blood vessel 6 are bent, and the length L5 of the intermediate portion 172 of the plug, the plug 150 will strive to continue movement along a straighter path as much as possible, which can cause the plug proximal end 154 to contact one side of the inner surface of the sheath 40 and act as a fulcrum around which the plug 150 may slightly pivot. The plug 150 preferably retains its shape and outer diameter D5, for example by virtue of the stopper arms 140 preventing it from recompressing in response to resistive forces applied thereto by the sheath and the surrounding anatomy, allowing it to act as a relatively rigid body that cannot be bent along at least the intermediate portion length L5.

This slight angular movement of the plug 150 can cause its central axis C2 to be no longer colinear with the central axis C1 of the sheath 40, as shown in FIG. 11B, forcing the expanded plug 150 to assume a somewhat angular orientation within the sheath lumen 50, with respect to the central axis C1 of the sheath. In such a case, one end of the intermediate portion 172 (e.g., some of third bending points 166C) can contact one side of the inner surface of the sheath 40, shown as the lower-right corner in the illustrated example, while the other end of the intermediate portion 172 (e.g., some of second bending points 166B) can contact the opposite side of the inner surface of the sheath 40, shown as the upper-left corner in the illustrated example. This can have the effect of advantageously forcing local straightening of the sheath 40 and/or the blood vessel 6 at such bent regions, as well as locally expanding the sheath to a diameter D8, dictated by the radial distance between the contact points of the intermediate portion 172 with the interior wall of the sheath on both opposite sides thereof, wherein D8 will thus be greater than the Diameter D7 to which the sheath 40 is locally expanded at unbent (or less bent) regions thereof.

Thus, utilization of a plug 150 having intermediate portion 172 with a uniform diameter along an intermediate portion length L5, is advantageous over alternative configurations by which an intermediate portion of the plug could assume a bent, curved, pointed, or any other configuration that has a non-uniform diameter along its axial length, particularly for straightening relatively bent portions of the sheath 40 when passing through highly curved regions of the patient's vasculature, as well as increasing the sheath's expanded diameter at such regions. In some examples, the intermediate portion length L5 is at least as great as half the second or expanded diameter D5 of the plug 150. In some examples, the intermediate portion length L5 is at least as great as the second or expanded diameter D5 of the plug 150. In some examples, the intermediate portion length L5 is at least 1.5 times greater than the diameter D5. In some examples, the intermediate portion length L5 is at least 2 times greater than the diameter D5. In some examples, the intermediate portion length L5 is at least 3 times greater than the diameter D5. In some examples, the intermediate portion length L5 is at least as great as the outer diameter D9 of the outer shaft 110. In some examples, the second plug diameter D5 greater than the D9 by at least 20% of D9. In some examples, the second plug diameter D5 greater than the D9 by at least 30% of D9. In some examples, the second plug diameter D5 greater than D9 by at least 50% of D9. In some examples, the second plug diameter D5 greater than D9 by at least 75% of D9.

While diameters of the sheath in either expanded or un-expanded states thereof, including any of its natural diameter D1, or expanded diameters D2, D6, D7 or D8, are shown as external diameters of the sheath throughout the drawings, it is to be understood that any of such diameters is similarly proportional to a corresponding internal diameter of the sheath 40 (i.e., diameter of the sheath lumen 50), which is equal to the illustrated external diameter from which twice the thickness of the wall of the sheath is subtracted. For example, any reference to a natural diameter D1 of the sheath, while illustrated as the external diameter of the sheath, is proportional to the internal natural diameter of the sheath, which can be the diameter of the sheath lumen 50 illustrated in FIG. 4.

As described above, the second plug diameter D5 is a uniform diameter along an intermediate portion length L5 of the intermediate portion of the plug, which is greater than the first plug diameter, at least as measured for the same intermediate portion. In some examples, the second plug diameter D5 greater than the first plug diameter by at least 20% of the first plug diameter. In some examples, the second plug diameter D5 greater than the first plug diameter by at least 30% of the first plug diameter. In some examples, the second plug diameter D5 greater than the first plug diameter by at least 50% of the first plug diameter. In some examples, the second plug diameter D5 greater than the first plug diameter by at least 75% of the first plug diameter.

In some examples, the first plug diameter can correspond to the outer diameter D9 of the outer shaft 110 of the introducer, such as being within a range of 15% of the diameter D9, resulting in the second plug diameter D5 being greater than the outer diameter D9 of the outer shaft 110. In some examples, the diameter D9 of the outer shaft is 14 F, and the second plug diameter D5 is greater than 14 F. In some examples, the diameter D9 of the introducer's outer shaft is 2 F, and the second plug diameter D5 is greater than 2 F. In some examples, the diameter D9 of the introducer's outer shaft is 4 F, and the second plug diameter D5 is greater than 4 F. In some examples, the diameter D9 of the introducer's outer shaft is 6 F, and the second plug diameter D5 is greater than 6 F. In some examples, the diameter D9 of the introducer's outer shaft is 8 F, and the second plug diameter D5 is greater than 8 F. In some examples, the diameter D9 of the introducer's outer shaft is 10 F, and the second plug diameter D5 is greater than 10 F. In some examples, the diameter D9 of the introducer's outer shaft is 12 F, and the second plug diameter D5 is greater than 12 F. In some examples, the diameter D9 of the introducer's outer shaft is 16 F, and the second plug diameter D9 is greater than 16 F. In some examples, the diameter D9 of the introducer's outer shaft is 18 F, and the second plug diameter D5 is greater than 18 F. In some examples, the diameter D9 of the introducer's outer shaft is 20 F, and the second plug diameter D5 is greater than 20 F. In some examples, the diameter D9 of the introducer's outer shaft is 22 F, and the second plug diameter D5 is greater than 22 F. In some examples, the diameter D4 of the introducer's outer shaft is 24 F, and the second plug diameter D5 is greater than 24 F. In some examples, the diameter D9 of the introducer's outer shaft is 26 F, and the second plug diameter D5 is greater than 26 F. In some examples, the diameter D9 of the introducer's outer shaft is 28 F, and the second plug diameter D5 is greater than 28 F. In other examples, the diameter D9 of the introducer's outer shaft is any suitable diameter as long as the second plug diameter D5 is greater than D9.

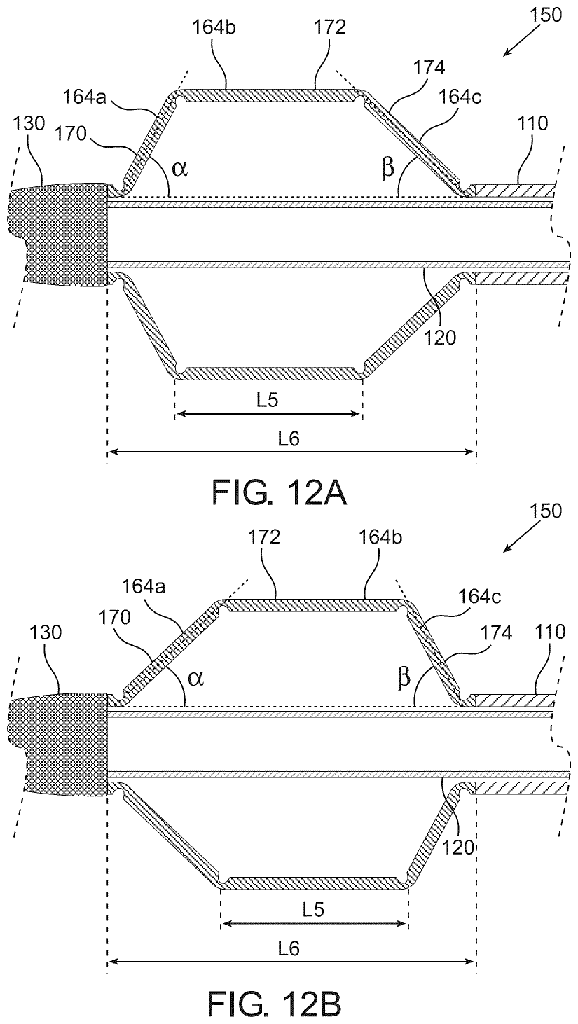

While FIGS. 8A-9 illustrate one example of the distal portion 170 and the proximal portion 174 similarly shaped to have a similar degree of taper in the expanded configuration of the plug 150, in some examples, the plug 150 can be designed to expand to an axially asymmetrical shape, in which the distal portion 170 and the proximal portion 174 have non-identical degrees of taper. FIGS. 12A-12B illustrate two such examples. The degree of taper of each of the portions 170, 174 can be referred to as a draft angle, which can be a measure of the angle between the plug's central axis C2 and a line tangent to the outer side 156 of each such portion. Specifically, the distal portion 170 can exhibit a first draft angle α, which can be defined between a first strut sections 164a and the central axis C2 or any axis parallel thereto, and the proximal portion 174 can exhibit a second draft angle β, which can be defined between a third strut sections 164c and the central axis C2 or any axis parallel thereto. FIG. 12A shows one example of a plug 150 shown in an expanded configuration, wherein the second draft angle β is more acute than the first draft angle α. FIG. 12B shows an example in which the first draft angle α is more acute than the second draft angle β.

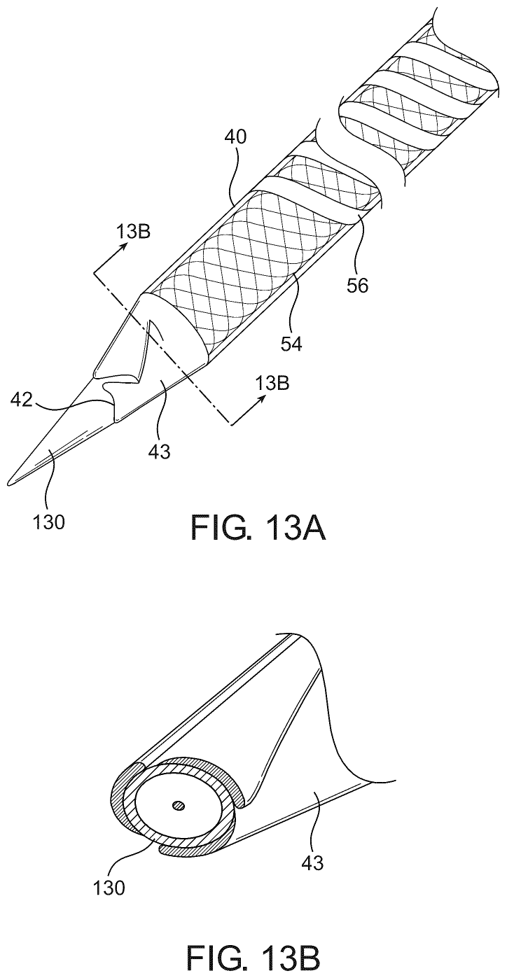

FIGS. 13A-13B show an example sheath 40 including a distal end portion 43, which can be an extension of an outer cover extending longitudinally along the sheath 40 in the proximal direction. FIG. 13A shows a distal end portion 43 folded around an introducer 100 (in the crimped and collapsed configuration), for example around the introducer's nosecone 130. FIG. 13B shows a cross section of the distal end portion 43 folded around the introducer 100 (in the crimped and collapsed configuration). The distal end portion 43, which terminates at sheath distal end 42, can be formed of, for example, one or more layers of a similar or the same material used to form the outer layer 58 of the sheath 40. In some examples, the distal end portion 43 includes an extension of the outer layer 58 of the sheath 40, with or without one more additional layers added by separate processing techniques. The distal end portion 43 can include anywhere from 1 to 8 layers of material (including 1, 2, 3, 4, 5, 6, 7, and 8 layers of material). In some examples, the distal end portion comprises multiple layers of a Dyneema® material. The distal end portion 43 can extend distally beyond a longitudinal portion of the sheath that includes braided layer 54 and elastic layer 56. In fact, in some examples, the braided layer 54 may extend distally beyond the elastic layer 56, and the distal end portion 43 may extend distally beyond both the braided layer 54 and elastic layer 56, as shown in FIGS. 13A-13B.

The distal end portion 43 may have a smaller collapsed diameter than the more proximal portions of the sheath, giving it a tapered appearance. This smooths the transition between the introducer 100 and the sheath 40, ensuring that the sheath 40 does not get lodged against the tissue during insertion into the patient. The smaller collapsed diameter can be a result of multiple folds (for example, 1, 2, 3, 4, 5, 6, 7, or 8 folds) positioned circumferentially (evenly or unevenly spaced) around the distal end portion. For example, a circumferential segment of the distal end portion 43 can be brought together and then laid against the adjacent outer surface of the distal end portion 43 to create an overlapping fold. In the collapsed configuration, the overlapping portions of the fold extend longitudinally along the distal end portion 43. Exemplary folding methods and configurations are described in U.S. Pat. Nos. 10,792,471 and 10,327,896, each of which are hereby incorporated by reference in their entireties. Scoring can be used as an alternative, or in addition to folding of the distal end portion 43. Both scoring and folding of the distal end portion 43 allow for the expansion of the distal end portion 43 upon the passage of the expanded plug 150, and ease the passage through and retraction of the delivery apparatus 10 back into the sheath 40 once the procedure is complete. In some examples, the distal end portion 43 of the sheath 40 (and/or of the nosecone 130 of the introducer) can decrease from the initial diameter of the sheath (e.g., 8 mm) to 3.3 mm (10 F), and may decrease to the diameter of a guidewire 32, allowing the sheath 40 and/or the introducer 100 to run on a guidewire 32.

In some examples, a distal end portion 43 can be added, the sheath 40 and tip can be crimped, and the crimping of the distal end portion 43 and sheath 40 can be maintained, by the following method. As mentioned above, the distal end portion 43 can be an extension of the outer layer 58 of the sheath 40. It can also be a separate, multilayer tubing that is heat bonded to the remainder of the sheath 40 prior to the tip crimping processing steps.

In some examples, the separate, multilayer tubing is heat bonded to a distal extension of the outer layer 58 of the sheath 40 to form the distal end portion 43. For crimping of the sheath 40 after tip attachment, the sheath 40 is heated on small mandrel. The distal end portion 43 can be folded around the mandrel to create the folded configuration shown in FIG. 13A. The folds can be added to the distal end portion 43 prior to the tip crimping process, or at an intermediate point during the tip crimping process. In some examples, the small mandrel can be from about 2 millimeters to about 4 millimeters in diameter (including about 2.2 millimeters, about 2.4 millimeters, about 2.6 millimeters, about 2.8 millimeters, about 3.0 millimeters, about 3.2 millimeters, about 3.4 millimeters, about 3.6 millimeters, about 3.8 millimeters and about 4.0 millimeters).

The heating temperature will be lower than the melting point of the material used. This can cause the material to shrink on its own to a certain extent. For example, in an example sheath where Dyneema® materials are utilized as part of the sheath 40 outer layer 58 and/or distal end portion 43 materials, a sheath crimping process begins by heating the sheath 40 on a 3 millimeter mandrel to about 125 degrees Celsius (lower than Dyneema® melting point of about 140 degrees Celsius). This causes the sheath 40 to crimp itself to about a 6 millimeter outer diameter. At the distal end portion 43, a higher temperature can be applied (for example, from about 145 degrees Celsius to about 155 degrees Celsius for Dyneema® material) causing the layers of material to melt together in the folded configuration shown in FIG. 13A. The bonds at the distal end portion 43 induced by the high temperature melting step will still be weak enough to be broken by a passing expanded plug.

In some examples, a method of utilizing introducer device assembly 20 can include passing the plug 150 in its compacted configuration past the sheath distal end 42, expanding the plug 150, and then retracting the expanded plug 150 back into the sheath 40 and pulling it along the central axis C1 of the sheath to facilitate sheath expansion, as described above. In such cases, it may be desirable to form the proximal portion 174 to exhibit an acute second draft angle β that will facilitate smooth movement of the plug 150 during retraction thereof through the sheath lumen 50. In some examples, the second draft angle β is less than 45 degrees. In some examples, the second draft angle β is less than 30 degrees. While it is important for the proximal portion 174 to exhibit a relatively acute second draft angle β when the plug is designed for retraction through the sheath 40, the first draft angle α is of less importance if no forward movement takes place, and may be less acute. Thus, when the plug 150 is designed for retraction through the sheath 40, it can optionally assume the shape illustrated in FIG. 12A. Utilizing a plug made of relatively rigid materials, such as struts or frames, allows it to advantageously retain a designed draft angle (such as any of the second draft angle β or the first draft angle α) during axial plug movement through the sheath 40, which is much more difficult for other less-rigid plug designs, such as plug that may be formed as inflatable balloons that allow only limited control over the draft angles at each end.

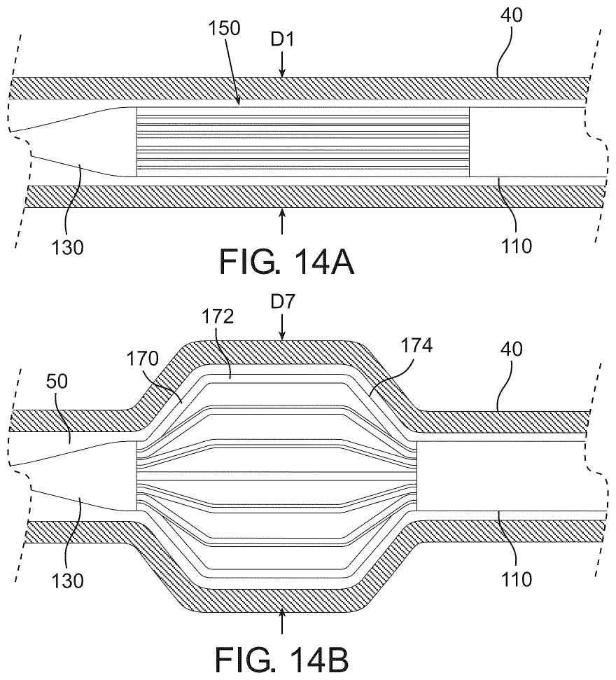

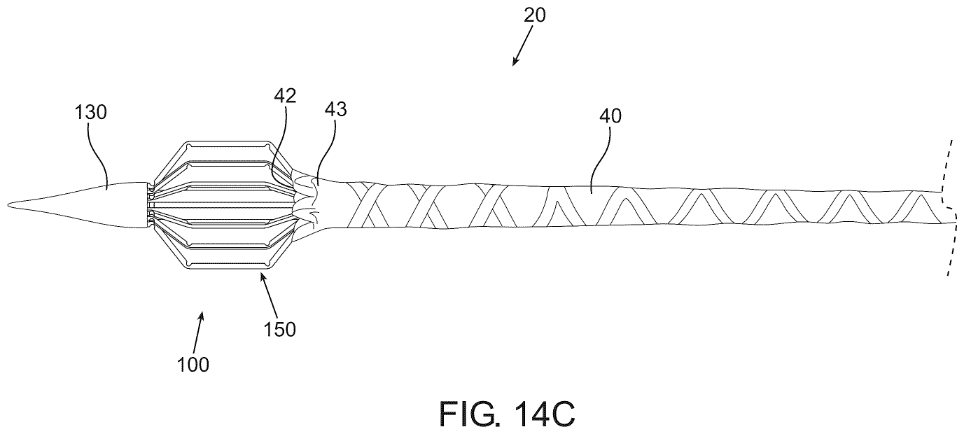

In some examples, a method of utilizing introducer device assembly 20 can include positioning the plug 150 in its compacted configuration within the sheath 40, proximal to sheath distal end 42, and expanding it within the sheath, and pushing it distally towards and past the sheath distal end 42. FIG. 14A shows a portion of an introducer device assembly 20 with the plug positioned within the sheath lumen 50, proximal to the sheath distal end 42, and expanded as shown in FIG. 14B while still positioned in lumen 50. In some examples, the plug 150 can be expanded when positioned at a proximal portion of the sheath 40, and then pushed in a distal direction towards and past the sheath distal end 42 to expand the sheath 40, optionally with its distal end portion 43. In some examples, the plug 150 can be expanded at a relatively distal portion of the sheath 40, which is still proximal to the sheath distal end 42, and optionally also proximal to the distal end portion 43 of the sheath, at which point it can be expanded and pushed distally to expand the distal end portion 43, as shown for example in FIG. 14C. When the expanded plug 150 is passed through the distal end portion 43 of the sheath 40, the distal end portion 43 can unfold and expand, after which the plug 150 can be optionally pulled back therethrough into the sheath 40, and pulled further in a proximal direction to further expand the remainder portion of the sheath 40.

In some examples, the plug 150 may be designed mainly to expand the distal end portion 43 of the sheath 40, for example by being distally pushed in its expanded configuration therethrough, without necessarily needing to further expand other portions of the sheath 40 (though it may be still utilized to do both). In such cases, it may be desirable to form the distal portion 170 to exhibit an acute first draft angle α that will facilitate smooth movement of the plug 150 when distally pushed through the distal end portion 43 of the sheath 40. In some examples, the first draft angle α is less than 45 degrees. In some examples, the first draft angle α is less than 30 degrees. While it is important for the distal portion 170 to exhibit a relatively acute first draft angle α when the plug is designed to be pushed through the distal end portion 43 of the sheath 40, the second draft angle β is of less importance if no backwards movement takes place, and may be less acute. Thus, when the plug 150 is designed to be pushed through the distal end portion 43 of the sheath 40, it can optionally assume the shape illustrated in FIG. 12B.

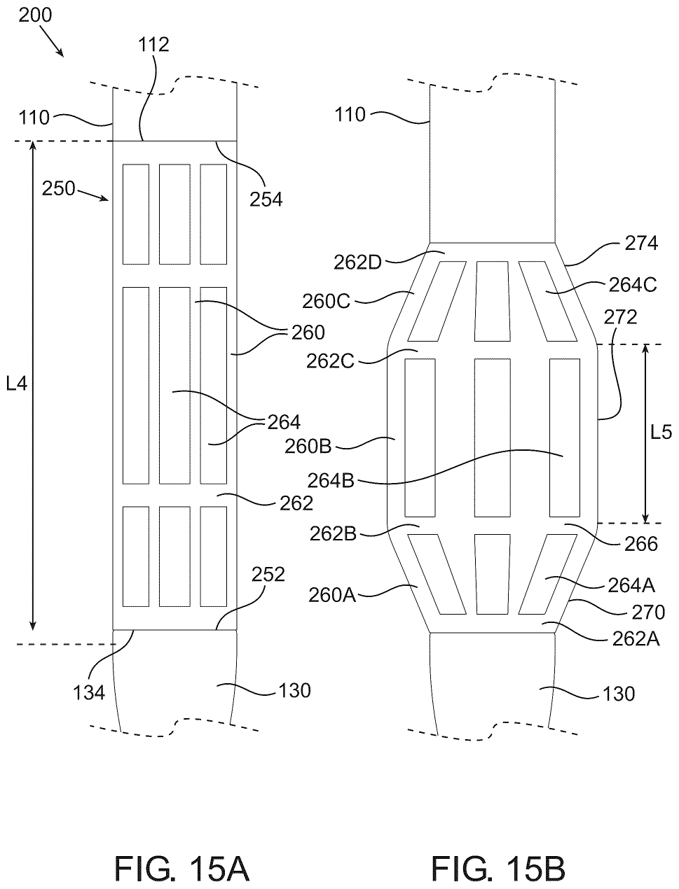

FIGS. 15A-17B illustrate variations of examples of introducers equipped with expendable plugs that form a linear intermediate portion in their expanded configurations. The features of the examples of FIGS. 15A-17B may be utilized with or applied to the examples of FIGS. 1-14C, and the features of the examples of FIGS. 1-14C may be utilized or applied to the examples of FIGS. 15A-17B.

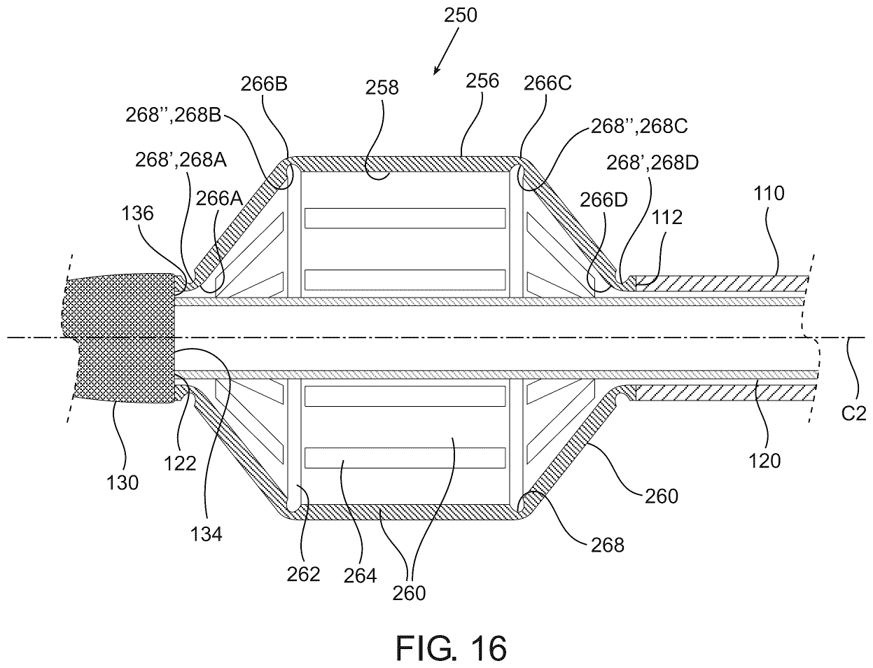

FIGS. 15A-16 illustrate an example of an introducer 200, which can be structurally and functionally similar to introducer 100, with like numbers referring to like components, except that it includes an expandable plug 250 in the form of an expandable frame comprising axial support sections and circumferential support sections. The expandable plug 250 is movable between a compacted configuration, shown in FIG. 15A, and an expanded configuration, shown in FIG. 15B. The expandable plug 250 has a plug distal end 252 and a plug proximal end 254, and defines a plug compacted length L4 extending from the plug distal end 252 to the plug proximal end 254 in a compacted configuration thereof. The plug 250 can be attached at its distal end 252, to the nosecone proximal end 134. The plug can be further attached at its proximal end 254 to the outer shaft distal end 112. Thus, as shown, the plug 250 is disposed around a distal portion of the inner shaft 120, and extends between the nosecone 130 and the outer shaft 110.

The plug 250 can define an inner side 258 facing the central axis C2, and an opposite outer side 256 facing away from the central axis C2. In some examples, the outer side 256 can include a hydrophilic coating to reduce friction between the sheath 40 and the plug 250. In some examples, the hydrophilic coating includes a material with a low coefficient of friction.

The expandable plug 250 includes a plurality of axial support sections 260 which are parallel, in the compacted configuration, to the central axis C2 of the plug, and a plurality of circumferential support sections 262, extending around a circumference of the plug, for example along planes orthogonal to main axis C2. The axial support sections 260 and the circumferential support sections 262 intersect with each other, forming slots 264 therebetween. In one example, the slots 264 can be rectangular, as illustrated. In other examples, the slots may be formed to have any other shape. The axial support sections 260 and the circumferential support sections 262 can be formed of a variety of materials and in a variety of shapes, as long as the shape and structure is sufficiently strong to cause local expansion of the sheath 40. For example, each axial support section 260 and/or circumferential support section 262 can be formed of a stiff polymeric or metallic material. In addition, the expandable plug 250 can include any variety of number of axial support sections 260 and circumferential support sections 262, so long as they are of sufficient number, strength, and/or shape so as to provide sufficient force to an inner surface of the sheath 40 (e.g., an inner surface of inner layer 52) to support local expansion of the sheath 40.

The plug 250 includes at least four circumferential support sections 262, axially spaced from each other. Each axial support section 260 axially extends between two adjacent circumferential support sections 262, such that at least three axial support sections 260 extend between the plug proximal end 254 and the plug distal end 252 at various circumferential positions around the plug 250. Thus, each slot 264 is confined between two axial support sections 260 and two circumferential support sections 262. The axial support sections 260 and the circumferential support sections 262 intersect at bending points 266, which can include, in some examples, notches 268, as shown for example in FIG. 13.

The circumferential support sections 262 in the illustrated example include a first circumferential support section 262A at the plug distal end 252, optionally affixed (e.g., glued, welded, fastened by the use of suitable fasteners, and the like) to the nosecone 130, such as to the nosecone proximal end 134, a second circumferential support section 262B proximal to the first circumferential support section 262A, a third circumferential support section 262C proximal to the second circumferential support section 262B, and a fourth circumferential support section 262D at the plug proximal end 254, which can be affixed to the outer shaft 110, such as to the outer shaft distal end 112, in a similar manner described for the first circumferential support section 262A.

Each axial line of support sections 260 extending between the ends of the plug 250 at a specific circumferential position includes: a first axial support section 260A extending between the first circumferential support section 262A and the second circumferential support section 262B, a second axial support section 260B extending between the second circumferential support section 262B and the third circumferential support section 262C, and a third axial support section 260C extending between the third circumferential support section 262C and the fourth circumferential support section 262D.

A first bending point 266A is formed at the junction between the first circumferential support section 262A and the first axial support section 260A. A second bending point 266B is formed at the junction between the first axial support section 260A, the second axial support section 260B, and the second circumferential support section 262B. A third bending point 266C is formed at the junction between the second axial support section 260B, the third axial support section 260C, and the third circumferential support section 262C. A fourth bending point 266D is formed at the junction between the fourth axial support section 260D and the fourth circumferential support section 262D.

The plug 250 is configured to transition from a compacted configuration, in which the plug 250 has a first plug diameter, to an expanded configuration, in which the plug 250 has a second plug diameter that is greater than the first plug diameter. The plug 250 is shown in FIG. 12A in a compacted or un-expanded configuration, in which the plug 250 is flattened. In this configuration, all of the axial support sections 260 can be substantially parallel to the central axis C2 of the plug, and all circumferential support sections 262 can have the same diameter, which is the first plug diameter. The plug 250 may transition to an expanded or deployed configuration, as shown in FIG. 12B, in which it has a plug second diameter D5 (not marked in FIG. 15B or 16, but similar to diameter D5 marked in FIG. 9), which is greater than the plug first diameter, and greater than the natural diameter D1 of the sheath 40. At least one axial support section 260 is spaced away from the inner shaft 120 in this configuration, to a greater extent than in the compacted configuration shown in FIG. 15A.

The plug 250 in the compacted configuration may be configured to fit within the sheath lumen 50. The plug 250 may have a plug first diameter that is sized to fit within the sheath lumen 50. The first plug diameter can be, in some examples, substantially equal to or less than the diameter D4 of the nosecone proximal end 144. The first plug diameter can be, in some examples, substantially equal to or less than the diameter D9 of the outer shaft 110. Thus, the plug 250 in the compacted configuration may fit within the sheath lumen 50 without increasing the size of the lumen 50 or otherwise expanding the sheath 40.

In some examples, notches 268 are formed on one side of the bending points 266, which can be either the outer side 256 or the inner side 258. Notching the appropriate bending points 266 facilitates the bending between adjacent axial support sections 260 to form a desired expanded shape. In the example illustrated in FIG. 16, four notches 268 are formed at bending points 266 at each axial line of axial support sections 260 extending at various circumferential positions between the plug distal end 252 and the plug proximal end 254. Specifically, each first bending point 266A can include a first notch 268A, each second bending point 266B can include a second notch 268B, each third bending point 266C can include a third notch 268C, and each fourth bending point 266D can include a fourth notch 268D.

The various notches 268 can include notches 268′ formed at the outer side 256 and notches 268″ formed at the inner side 258, to facilitate a desired direction of bending at the corresponding bending points 266. In the example illustrated in FIG. 13, the notches disposed between axial support sections 260 and circumferential support sections 262 at both ends 252, 254 of the plug are notches 268′ formed at the outer side 256, and those disposed between two subsequent axial support sections 260 are notches 268″ formed at the inner side 258. Specifically, the first bending point 266A and fourth bending point 266D, which include notches 268′ formed at the outer side 256, allow the first axial support section 260A and third axial support section 260C to bend radially outward, away from the respective first circumferential support section 262A and fourth circumferential support section 262D. Similarly, the notches 268″ at second bending point 266B and third bending point 266C, disposed on both sides of the second axial support section 260B, allow it to remain relatively parallel with central axis C2, while axial support sections 260A and 260C on both sides thereof assume an angled configuration relative to the axis C2 in the expanded configuration. This eventually allows radial displacement of the second axial support sections 260B away from axis C2, while remaining substantially parallel with respect to the axis C2.

The plug 250 can be expanded via axial movement of the inner shaft 120 and outer shaft 110 relative to each other, in a manner that approximates the plug distal end 252 and plug proximal end 254, from a distance or plug length L4 in the collapse configuration (see FIG. 16A), to a shorter distance or plug length L6 in the expanded configuration (not separately marked in FIG. 16B, but similar to length L6 marked in FIG. 8B). This can be accomplished according to any example described above for facilitating expansion of the plug 150, mutatis mutandis. The introducer 200 can further include one or more stopper arms 140, implemented and utilized according to any examples described above with respect to introducer 100.

The plug 250 can define a distal portion 270 comprising the first axial support sections 260A, an intermediate portion 272 comprising the second axial support sections 260B, and a proximal portion 174 comprising the third axial support sections 260C. The distal and proximal portions 270 and 274 can transition from a relatively cylindrical shape in the compacted configuration of FIG. 15A, to tapering shapes in the expanded configuration extending from a wider diameter to the narrower diameter (e.g., from diameter D5 to diameter D4 and/or D9) as shown in FIGS. 15B and 16, such that the projected length of these portions 270, 274 over central axis C2, for example, shortens when transitioning from the compacted to the expanded configuration. In contrast, the projected length L5 of intermediate portion 272, which is the length of second axial support sections 260B in the illustrated example, remains constant during both the compacted and the expanded configuration of the plug 250. This ensures that in the expanded configuration, the intermediate portion 272 of the plug 250 retains an intermediate portion length L5 along which it exhibits a uniform diameter (e.g., the second plug diameter D5). The distal portion 270 and the proximal portion 274 exhibit first and second draft angles α and β, respectively, which can be similar or different from each other, as described for such draft angles with respect to expandable plug 150 above.

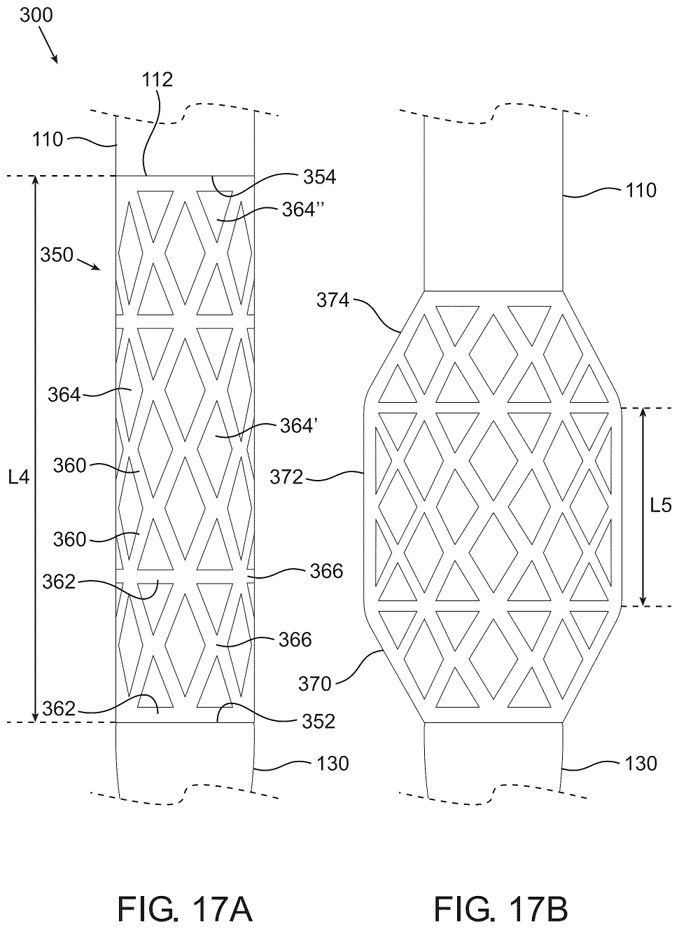

FIGS. 17A-17B illustrate another example of an introducer 300, which can be structurally and functionally similar to introducer 100, with like numbers referring to like components, except that it includes an expandable plug 350 in the form of an expandable frame comprising intersecting angled struts. The expandable plug 350 is movable between a compacted configuration, shown in FIG. 17A, and an expanded configuration, shown in FIG. 17B. The expandable plug 350 has a plug distal end 352 and a plug proximal end 354, and defines a plug compacted length L4 extending from the plug distal end 352 to the plug proximal end 354 in a compacted configuration thereof. The plug 350 can be attached at its distal end 352, to the nosecone proximal end 134. The plug can be further attached at its proximal end 354 to the outer shaft distal end 112. Thus, as shown, the plug 350 is disposed around a distal portion of the inner shaft 120, and extends between the nosecone 130 and the outer shaft 110.

In some examples, an outer surface of the plug 350 can include a hydrophilic coating to reduce friction between the sheath 40 and the plug 350. In some examples, the hydrophilic coating includes a material with a low coefficient of friction.

The expandable plug 350 includes a plurality of angled struts 360 which are angled with respect to the central axis C2, and a plurality of circumferential struts 362, extending around a circumference of the plug, for example along planes orthogonal to main axis C2. The angled struts 360 intersect at junctions 366 with each other, and in some examples, with the circumferential struts 362, forming cell openings 364 therebetween. In one example, the cell opening 364 can be diamond shaped when bound between four angled struts 360, and triangular when bound between a circumferential strut 362 and two angled struts 360, as illustrated. In other examples, the cell openings may be formed to have any other shape. The angled struts 360 and the circumferential struts 362 can be formed of a variety of materials and in a variety of shapes, as long as the shape and structure is sufficiently strong to cause local expansion of the sheath 40. For example, each axial strut 360 and/or circumferential strut 362 can be formed of a stiff polymeric or metallic material. In addition, the expandable plug 350 can include any variety of number of axial struts 360 and circumferential struts 362, so long as they are of sufficient number, strength, and/or shape so as to provide sufficient force to an inner surface of the sheath 40 (e.g., an inner surface of inner layer 52) to support local expansion of the sheath 40.

The angled struts 360 are pivotable or bendable relative to each other, and when connected to circumferential struts 362, relative to the circumferential struts as well, to permit radial expansion of the plug 350. For example, the plug 350 can be formed (e.g., via laser cutting, electroforming or physical vapor deposition) from a single piece of material (e.g., a metal tube). As such, the plug distal end 352 and the plug proximal end 354 can move axially parallel to the central axis C2 as the plug 350 is radially expanded.

The plug 350 is configured to transition from a compacted configuration, in which the plug 350 has a first plug diameter, to an expanded configuration, in which the plug 350 has a second plug diameter that is greater than the first plug diameter. The plug 350 is shown in FIG. 17A in a compacted or un-expanded configuration, in which the plug 350 is flattened. The plug 350 may transition to an expanded or deployed configuration, as shown in FIG. 17B, in which it has a plug second diameter D5 (not marked in FIGS. 17B, but similar to diameter D5 marked in FIG. 9), which is greater than the plug first diameter, and greater than the natural diameter D1 of the sheath 40.

The plug 350 in the compacted configuration may be configured to fit within the sheath lumen 50. The plug 350 may have a plug first diameter that is sized to fit within the sheath lumen 50. The first plug diameter can be, in some examples, substantially equal to or less than the diameter D4 of the nosecone proximal end 144. The first plug diameter can be, in some examples, substantially equal to or less than the diameter D9 of the outer shaft 110. Thus, the plug 350 in the compacted configuration may fit within the sheath lumen 50 without increasing the size of the lumen 50 or otherwise expanding the sheath 40.

The plug 350 can be expanded via axial movement of the inner shaft 120 and outer shaft 110 relative to each other, in a manner that approximates the plug distal end 352 and plug proximal end 354, from a distance or plug length L4 in the collapse configuration (see FIG. 17A), to a shorter distance or plug length L6 in the expanded configuration (not separately marked in FIG. 17B, but similar to length L6 marked in FIG. 8B). This can be accomplished according to any example described above for facilitating expansion of the plug 150, mutatis mutandis. The introducer 300 can further include one or more stopper arms 140, implemented and utilized according to any examples described above with respect to introducer 100.

The plug 350 can include at least two rungs of circumferential struts 362: one rung at the plug distal end 352, optionally affixed (e.g., glued, welded, fastened by the use of suitable fasteners, and the like) to the nosecone 130, such as to the nosecone proximal end 134, and another rung at the plug proximal end 354, which can be similarly affixed to the outer shaft 110, such as to the outer shaft distal end 112. In some examples, the plug can further include two additional intermediate rungs of circumferential struts 362, axially spaced from each other, disposed between the plug distal end 352 and the plug proximal end 354.

The plug 350 can define a distal portion 370 comprising angled struts 360 extending between the rung of the circumferential struts 362 at the plug distal end 352 and the first intermediate rung of circumferential struts 362 proximal thereto, an intermediate portion 372 comprising angled struts 360 extending between both intermediate rungs of circumferential struts 362, and a proximal portion 374 angled struts 360 extending between the intermediate rung of the circumferential struts 362 at the proximal end of the central intermediate 372 and the rung of the circumferential struts 362 at the plug proximal end 354. The distal and proximal portions 370 and 374 can transition from a relatively cylindrical shape in the compacted configuration of FIG. 17A, to tapering shapes in the expanded configuration extending from a wider diameter to the narrower diameter (e.g., from diameter D5 to diameter D4 and/or D9) as shown in FIG. 17B, such that the projected length of these portions 370, 374 over central axis C2, for example, shortens when transitioning from the compacted to the expanded configuration. In contrast, the plug 350 is designed such that the shape of intermediate portion 372 remains cylindrical in the expanded configuration of the plug 350, exhibiting a uniform diameter (e.g., plug second diameter D5) along an intermediate portion length L5.

The rungs of circumferential struts 362 can be designed to facilitate bending of angled struts extending in axially opposing directions from the junctions 366, relative to each other. Thus, the addition of intermediate rungs of circumferential struts 362 at both ends of the intermediate portion 372 can assist in retaining a relatively cylindrical intermediate portion 372 having a uniform diameter along the intermediate portion length L5 in the expanded configuration. Nevertheless, it is to be understood that the plug can include other features by which a cylindrical shape of the intermediate portion 372 can be retained in the expanded configuration, such as by designing the plug to include varying sizes of cell opening 364 and/or varying thicknesses and/or widths of angled struts at different regions of the plug. The distal portion 370 and the proximal portion 374 exhibit first and second draft angles α and β, respectively, which can be similar or different from each other, as described for such draft angles with respect to expandable plug 150 above.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. An introducer, comprising:
a nosecone tapering from a nosecone proximal end to a nosecone distal tip;
an inner shaft having an inner shaft distal end attached to the nosecone;
an outer shaft disposed around the inner shaft, the outer shaft having an outer shaft distal end proximal to the inner shaft distal end, and an outer shaft proximal end; and
a plug disposed around the inner shaft, the plug having a plug distal end affixed to the nosecone, a plug proximal end affixed to the outer shaft, a distal portion extending proximally from the plug distal end, a proximal portion extending distally from the plug proximal end, and an intermediate portion between the distal portion and the proximal portion;
wherein the plug is configured to expand, upon approximation of the plug distal end and the plug proximal end to each other, from a plug first diameter in a compacted configuration of the plug, to a second plug diameter in an expanded configuration of the plug; and
wherein the intermediate portion exhibits a uniform diameter equal to the second plug diameter along an intermediate portion length thereof in the expanded configuration.

Example 2. The introducer of any example herein, particularly example 1, wherein the inner shaft and the outer shaft are axially movable relative to each other.

Example 3. The introducer of any example herein, particularly example 2, wherein movement of the inner shaft in a proximal direction relative to the outer shaft is configured to approximate the plug distal end to the plug proximal end.

Example 4. The introducer of any example herein, particularly example 2 or 3, wherein movement of the outer shaft in a distal direction relative to the inner shaft is configured to approximate the plug proximal end to the plug distal end.

Example 5. The introducer of any example herein, particularly any one of examples 1 to 4, wherein the plug is configured to slide, in its expanded configuration, in a proximal direction within a lumen of a sheath, so as to expand the sheath in the direction radially outward from a central axis of the sheath.

Example 6. The introducer of any example herein, particularly any one of examples 1 to 5, wherein the intermediate portion length in the expanded configuration is at least as great as half the second plug diameter.

Example 7. The introducer of any example herein, particularly any one of examples 1 to 5, wherein the intermediate portion length in the expanded configuration is at least as great the second plug diameter.

Example 8. The introducer of any example herein, particularly any one of examples 1 to 5, wherein the intermediate portion length in the expanded configuration is at least 1.5 times greater than the second plug diameter.

Example 9. The introducer of any example herein, particularly any one of examples 1 to 5, wherein the intermediate portion length in the expanded configuration is at least 2 times greater than the second plug diameter.

Example 10. The introducer of any example herein, particularly any one of examples 1 to 5, wherein the intermediate portion length in the expanded configuration is at least 3 times greater than the second plug diameter.

Example 11. The introducer of any example herein, particularly any one of examples 1 to 10, wherein the plug distal end is affixed to the nosecone proximal end.

Example 12. The introducer of any example herein, particularly any one of examples 1 to 11, wherein the plug proximal end is affixed to the outer shaft distal end.

Example 13. The introducer of any example herein, particularly any one of examples 1 to 13, wherein the distal portion is tapering in the expanded configuration, from the second plug diameter to a narrower diameter at the plug distal end.

Example 14. The introducer of any example herein, particularly any one of examples 1 to 13, wherein the proximal portion is tapering in the expanded configuration, from the second plug diameter to a narrower diameter at the plug proximal end.

Example 15. The introducer of any example herein, particularly any one of examples 1 to 14, wherein the second plug diameter is greater than the first plug diameter by at least 20% of the first plug diameter.

Example 16. The introducer of any example herein, particularly any one of examples 1 to 14, wherein the second plug diameter is greater than the first plug diameter by at least 30% of the first plug diameter.

Example 17. The introducer of any example herein, particularly any one of examples 1 to 14, wherein the second plug diameter is greater than the first plug diameter by at least 50% of the first plug diameter.

Example 18. The introducer of any example herein, particularly any one of examples 1 to 14, wherein the second plug diameter is greater than the first plug diameter by at least 75% of the first plug diameter.

Example 19. The introducer of any example herein, particularly any one of examples 1 to 18, wherein the outer shaft defines an outer diameter, and wherein the intermediate portion length in the expanded configuration is greater than the outer diameter of the outer shaft.

Example 20. The introducer of any example herein, particularly example 19, wherein the intermediate portion length in the expanded configuration is greater than the outer diameter of the outer shaft by at least 20% of the outer diameter of the outer shaft.

Example 21. The introducer of any example herein, particularly example 19, wherein the intermediate portion length in the expanded configuration is greater than the outer diameter of the outer shaft by at least 30% of the outer diameter of the outer shaft.

Example 22. The introducer of any example herein, particularly example 19, wherein the intermediate portion length in the expanded configuration is greater than the outer diameter of the outer shaft by at least 50% of the outer diameter of the outer shaft.

Example 23. The introducer of any example herein, particularly example 19, wherein the intermediate portion length in the expanded configuration is greater than the outer diameter of the outer shaft by at least 75% of the outer diameter of the outer shaft.

Example 24. The introducer of any example herein, particularly any one of examples 1 to 23, wherein the distal portion defines a first draft angle in the expanded configuration of the plug, and wherein the proximal portion defines a second draft angle in the expanded configuration of the plug.

Example 25. The introducer of any example herein, particularly example 24, wherein the second draft angle is more acute than the first draft angle.

Example 26. The introducer of any example herein, particularly example 24, wherein the first draft angle is more acute than the second draft angle.

Example 27. The introducer of any example herein, particularly any one of examples 24 to 26, wherein the second draft angle is less than 45 degrees.

Example 28. The introducer of any example herein, particularly any one of examples 24 to 26, wherein the second draft angle is less than 30 degrees.

Example 29. The introducer of any example herein, particularly any one of examples 24 to 28, wherein the first draft angle is less than 45 degrees.

Example 30. The introducer of any example herein, particularly any one of examples 24 to 28, wherein the leading first angle is less than 30 degrees.

Example 31. The introducer of any example herein, particularly any one of examples 1 to 30, wherein the plug comprises a plurality of struts longitudinally extending between the plug distal end and the plug proximal end.

Example 32. The introducer of any example herein, particularly example 31, wherein the plurality of struts are circumferentially spaced from each other.

Example 33. The introducer of any example herein, particularly example 31 or 32, wherein each strut of the plurality of struts comprises a plurality of strut sections, separated from each other by bending point.

Example 34. The introducer of any example herein, particularly example 33, wherein the plurality of strut sections of each strut comprises a first strut section, a second strut section, and a third strut section.

Example 35. The introducer of any example herein, particularly example 34, wherein the distal portion comprises the first strut sections, the intermediate portion comprises the second strut sections, and the proximal portion comprises the third strut sections.

Example 36. The introducer of any example herein, particularly example 34 or 35, wherein the second strut sections remain parallel to a central axis of the plug both in the compacted configuration and the expanded configuration.

Example 37. The introducer of any example herein, particularly any one of examples 34 to 36, wherein each strut of the plurality of struts further comprises a first strut end portion extending from the plug distal end to a corresponding bending point separating it from the corresponding first strut section, and a second strut end portion extending from the plug proximal end to another corresponding bending point separating it from the corresponding third strut section.

Example 38. The introducer of any example herein, particularly example 35, wherein the first strut end portions and the second strut end portions remain unmoved during the transition between the compacted configuration and the expanded configuration of the plug.

Example 39. The introducer of any example herein, particularly any one of examples 33 to 38, wherein each bending point comprises a notch.

Example 40. The introducer of any example herein, particularly example 39, wherein the notches disposed between each two adjacent strut sections are formed at an inner side of the struts.

Example 41. The introducer of any example herein, particularly example 37, wherein each bending point comprises a notch, and wherein the notches disposed between strut end portions and adjacent strut sections are formed at an outer side of the struts.

Example 42. The introducer of any example herein, particularly any one of examples 1 to 30, wherein the plug comprises a plurality of axial support sections and a plurality of circumferential support sections, wherein the plurality of axial support sections are parallel, in the compacted configuration, to a central axis of the plug, and wherein the plurality of circumferential support sections extend around a circumference of the plug.

Example 43. The introducer of any example herein, particularly example 42, wherein the plurality of axial support sections intersects with the plurality of circumferential support sections at bending points, forming a plurality of slots therebetween.

Example 44. The introducer of any example herein, particularly example 43, wherein the plurality of slots are rectangularly shaped.

Example 45. The introducer of any example herein, particularly any one of examples 42 to 44, wherein the plurality of circumferential support sections comprises a first circumferential support section at the plug distal end, a second circumferential support section proximal to the first circumferential support section, a third circumferential support section proximal to the second circumferential support section, and a fourth circumferential support section at the plug proximal end.

Example 46. The introducer of any example herein, particularly example 45, wherein the plurality of axial support sections comprises first axial support sections extending between the first circumferential support section and the second circumferential support section, second axial support sections extending between the second circumferential support section and the third circumferential support section, and third axial support sections extending between the third circumferential support section and the fourth circumferential support section.

Example 47. The introducer of any example herein, particularly example 46, wherein the distal portion comprises the first axial support sections, the intermediate portion comprises the second axial support sections, and the proximal portion comprises the third axial support sections.

Example 48. The introducer of any example herein, particularly example 46 or 47, wherein the second axial support sections remain parallel to the central axis of the plug both in the compacted configuration and the expanded configuration.

Example 49. The introducer of any example herein, particularly example 43, wherein each bending point comprises a notch.

Example 50. The introducer of any example herein, particularly example 49, wherein the notches disposed between each two adjacent axial support sections are formed at an inner side of the plug.

Example 51. The introducer of any example herein, particularly example 45, wherein each bending point comprises a notch, and wherein the notches disposed along the first circumferential support section and the fourth circumferential support section are formed at an inner side of the plug.

Example 52. The introducer of any example herein, particularly any one of examples 1 to 30, wherein the plug comprises a plurality of struts that comprises a plurality of angled struts and a plurality of circumferential struts, wherein the plurality of angled struts are angled with respect to a central axis of the plug, and wherein the plurality of circumferential struts extend around a circumference of the plug.

Example 53. The introducer of any example herein, particularly example 52, wherein the plurality of struts intersect with each other at junctions, forming a plurality of cell openings therebetween.

Example 54. The introducer of any example herein, particularly example 53, wherein the plurality of cell openings comprises diamond-shaped cell openings, each of which is bound between four angled struts.

Example 55. The introducer of any example herein, particularly example 53 or 54, wherein the plurality of cell openings comprises triangular cell openings, each of which is bound between two angled struts and one circumferential strut.

Example 56. The introducer of any example herein, particularly any one of examples 52 to 55, wherein the plurality of circumferential struts comprises a rung of circumferential struts at the plug distal end, a rung of circumferential struts at the proximal end, and two intermediate rungs of circumferential struts at both ends of the intermediate portion.

Example 57. The introducer of any example herein, particularly any one of examples 1 to 56, further comprising at least one stopper arm attached at a stopper proximal end to an outer surface of the inner shaft, and terminating with a stopper distal free end which is biased radially outward, wherein the stopper distal free end is axially positioned distal to the outer shaft proximal end when the plug is in the compacted configuration, and is axially positioned proximal to the outer shaft proximal end when the plug is in the expanded configuration.

Example 58. The introducer of any example herein, particularly example 57, wherein the stopper arm is retained in a folded configuration between the inner shaft and the outer shaft when the plug is in the compacted configuration.

Example 59. The introducer of any example herein, particularly example 57 or 58, wherein the stopper arm is biased radially outward such that the stopper distal free end abuts the outer shaft proximal end when the plug is in the expanded configuration.

Example 60. The introducer of any example herein, particularly example 59, wherein the stopper arm is configured to prevent distal movement of the inner shaft relative to the outer shaft, and/or proximal movement of the outer shaft relative to the inner shaft, when the plug is in the expanded configuration.

Example 61. The introducer of any example herein, particularly any one of examples 57 to 60, wherein the at least one stopper arm comprises a plurality of stopper arms.

Example 62. The introducer of any example herein, particularly any one of examples 1 to 61, wherein the outer shaft and the inner shaft are flexible.

Example 63. An introducer device assembly, comprising:
a sheath having a sheath distal end and a sheath lumen, wherein the sheath has a natural diameter in a non-expanded state thereof, and is expandable in a direction radially outward from a central axis of the sheath to a diameter which is greater than its natural diameter;
an introducer configured to axially move within the sheath lumen, the introducer comprising:
   a nosecone tapering from a nosecone proximal end to a nosecone distal tip;
   an inner shaft having an inner shaft distal end attached to the nosecone;
   an outer shaft disposed around the inner shaft, the outer shaft having an outer shaft distal end proximal to the inner shaft distal end, and an outer shaft proximal end; and
   a plug disposed around the inner shaft, the plug having a plug distal end affixed to the nosecone, a plug proximal end affixed to the outer shaft, a distal portion extending proximally from the plug distal end, a proximal portion extending distally from the plug proximal end, and an intermediate portion between the distal portion and the proximal portion;
wherein the plug is configured to expand, upon approximation of the plug distal end and the plug proximal end to each other, from a plug first diameter in a compacted configuration of the plug, to a second plug diameter in an expanded configuration of the plug; and
wherein the intermediate portion exhibits a uniform diameter equal to the second plug diameter along an intermediate portion length thereof in the expanded configuration.

Example 64. The assembly of any example herein, particularly example 63, wherein the inner shaft and the outer shaft are axially movable relative to each other.

Example 65. The assembly of any example herein, particularly example 64, wherein movement of the inner shaft in a proximal direction relative to the outer shaft is configured to approximate the plug distal end to the plug proximal end.

Example 66. The assembly of any example herein, particularly example 64 or 65, wherein movement of the outer shaft in a distal direction relative to the inner shaft is configured to approximate the plug proximal end to the plug distal end.

Example 67. The assembly of any example herein, particularly any one of examples 63 to 66, wherein the plug is configured to slide, in its expanded configuration, in a proximal direction within the sheath lumen, to expand the sheath in the direction radially outward from the central axis of the sheath.

Example 68. The assembly of any example herein, particularly any one of examples 63 to 67, wherein the intermediate portion length in the expanded configuration is at least as great as half the second plug diameter.

Example 69. The assembly of any example herein, particularly any one of examples 63 to 67, wherein the intermediate portion length in the expanded configuration is at least as great the second plug diameter.

Example 70. The assembly of any example herein, particularly any one of examples 63 to 67, wherein the intermediate portion length in the expanded configuration is at least 1.5 times greater than the second plug diameter.

Example 71. The assembly of any example herein, particularly any one of examples 63 to 67, wherein the intermediate portion length in the expanded configuration is at least 2 times greater than the second plug diameter.

Example 72. The assembly of any example herein, particularly any one of examples 63 to 67, wherein the intermediate portion length in the expanded configuration is at least 3 times greater than the second plug diameter.

Example 73. The assembly of any example herein, particularly any one of examples 63 to 72, wherein the plug distal end is affixed to the nosecone proximal end.

Example 74. The assembly of any example herein, particularly any one of examples 63 to 73, wherein the plug proximal end is affixed to the outer shaft distal end.

Example 75. The assembly of any example herein, particularly any one of examples 63 to 74, wherein the distal portion is tapering in the expanded configuration, from the second plug diameter to a narrower diameter at the plug distal end.

Example 76. The assembly of any example herein, particularly any one of examples 63 to 75, wherein the proximal portion is tapering in the expanded configuration, from the second plug diameter to a narrower diameter at the plug proximal end.

Example 77. The assembly of any example herein, particularly any one of examples 63 to 76, wherein the second plug diameter is greater than the first plug diameter by at least 20% of the first plug diameter.

Example 78. The assembly of any example herein, particularly any one of examples 63 to 76, wherein the second plug diameter is greater than the first plug diameter by at least 30% of the first plug diameter.

Example 79. The assembly of any example herein, particularly any one of examples 63 to 76, wherein the second plug diameter is greater than the first plug diameter by at least 50% of the first plug diameter.

Example 80. The assembly of any example herein, particularly any one of examples 63 to 76, wherein the second plug diameter is greater than the first plug diameter by at least 75% of the first plug diameter.

Example 81. The assembly of any example herein, particularly any one of examples 63 to 80, wherein the outer shaft defined an outer diameter, and wherein the minimal length of the intermediate portion in the expanded configuration is greater than the outer diameter of the outer shaft.

Example 82. The assembly of any example herein, particularly example 81, wherein the intermediate portion length in the expanded configuration is greater than the outer diameter of the outer shaft by at least 20% of the outer diameter of the outer shaft.

Example 83. The assembly of any example herein, particularly example 81, wherein the intermediate portion length in the expanded configuration is greater than the outer diameter of the outer shaft by at least 30% of the outer diameter of the outer shaft.

Example 84. The assembly of any example herein, particularly example 81, wherein the intermediate portion length in the expanded configuration is greater than the outer diameter of the outer shaft by at least 50% of the outer diameter of the outer shaft.

Example 85. The assembly of any example herein, particularly example 81, wherein the intermediate portion length in the expanded configuration is greater than the outer diameter of the outer shaft by at least 75% of the outer diameter of the outer shaft.

Example 86. The assembly of any example herein, particularly any one of examples 63 to 85, wherein the distal portion defines a first draft angle in the expanded configuration of the plug, and wherein the proximal portion defines a second draft angle in the expanded configuration of the plug.

Example 87. The assembly of any example herein, particularly example 86, wherein the second draft angle is more acute than the first draft angle.

Example 88. The assembly of any example herein, particularly example 86, wherein the first draft angle is more acute than the second draft angle.

Example 89. The assembly of any example herein, particularly any one of examples 86 to 88, wherein the second draft angle is less than 45 degrees.

Example 90. The assembly of any example herein, particularly any one of examples 86 to 88, wherein the second draft angle is less than 30 degrees.

Example 91. The assembly of any example herein, particularly any one of examples 86 to 90, wherein the first draft angle is less than 45 degrees.

Example 92. The assembly of any example herein, particularly any one of examples 86 to 90, wherein the first draft angle is less than 30 degrees.

Example 93. The assembly of any example herein, particularly any one of examples 63 to 92, wherein the sheath comprises a distal end portion terminating at the sheath distal end, wherein the distal end portion of the sheath comprises a tapered tip portion adapted for insertion into a body tissue in a non-expanded state of the sheath, and wherein the distal end portion of the sheath is configured to expand when the plug is pass therethrough in the expanded configuration of the plug.

Example 94. The assembly of any example herein, particularly any one of examples 63 to 93, wherein the plug comprises a plurality of struts longitudinally extending between the plug distal end and the plug proximal end.

Example 95. The assembly of any example herein, particularly example 94, wherein the plurality of struts are circumferentially spaced from each other.

Example 96. The assembly of any example herein, particularly example 94 or 95, wherein each strut of the plurality of struts comprises a plurality of strut sections, separated from each other by bending point.

Example 97. The assembly of any example herein, particularly example 96, wherein the plurality of strut sections of each strut comprises a first strut section, a second strut section, and a third strut section.

Example 98. The assembly of any example herein, particularly example 97, wherein the distal portion comprises the first strut sections, the intermediate portion comprises the second strut sections, and the proximal portion comprises the third strut sections.

Example 99. The assembly of any example herein, particularly example 97 or 98, wherein the second strut sections remain parallel to a central axis of the plug both in the compacted configuration and the expanded configuration.

Example 100. The assembly of any example herein, particularly any one of examples 97 to 99, wherein each strut of the plurality of struts further comprises a first strut end portion extending from the plug distal end to a corresponding bending point separating it from the corresponding first strut section, and a second strut end portion extending from the plug proximal end to another corresponding bending point separating it from the corresponding third strut section.

Example 101. The assembly of any example herein, particularly example 98, wherein the first strut end portions and the second strut end portions remain unmoved during the transition the compacted configuration to the expanded configuration of the plug.

Example 102. The assembly of any example herein, particularly any one of examples 96 to 101, wherein each bending point comprises a notch.

Example 103. The assembly of any example herein, particularly example 102, wherein the notches disposed between each two adjacent strut sections are formed at an inner side of the struts.

Example 104. The assembly of any example herein, particularly example 100, wherein each bending point comprises a notch, and wherein the notches disposed between strut end portions and adjacent strut sections are formed at an outer side of the struts.

Example 105. The assembly of any example herein, particularly any one of examples 63 to 96, wherein the plug comprises a plurality of axial support sections and a plurality of circumferential support sections, wherein the plurality of axial support sections are parallel, in the compacted configuration, to a central axis of the plug, and wherein the plurality of circumferential support sections extend around a circumference of the plug.

Example 106. The assembly of any example herein, particularly example 105, wherein the plurality of axial support sections intersect with the plurality of circumferential support sections at bending points, forming a plurality of slots therebetween.

Example 107. The assembly of any example herein, particularly example 106, wherein the plurality of slots are rectangularly shaped.

Example 108. The assembly of any example herein, particularly any one of examples 105 to 107, wherein the plurality of circumferential support sections comprises a first circumferential support section at the plug distal end, a second circumferential support section proximal to the first circumferential support section, a third circumferential support section proximal to the second circumferential support section, and a fourth circumferential support section at the plug proximal end.

Example 109. The assembly of any example herein, particularly example 108, wherein the plurality of axial support sections comprises first axial support sections extending between the first circumferential support section and the second circumferential support section, second axial support sections extending between the second circumferential support section and the third circumferential support section, and third axial support sections extending between the third circumferential support section and the fourth circumferential support section.

Example 110. The assembly of any example herein, particularly example 109, wherein the distal portion comprises the first axial support sections, the intermediate portion comprises the second axial support sections, and the proximal portion comprises the third axial support sections.

Example 111. The assembly of any example herein, particularly example 109 or 110, wherein the second axial support sections remain parallel to the central axis of the plug both in the compacted configuration and the expanded configuration.

Example 112. The assembly of any example herein, particularly example 106, wherein each bending point comprises a notch.

Example 113. The assembly of any example herein, particularly example 112, wherein the notches disposed between each two adjacent axial support sections are formed at an inner side of the plug.

Example 114. The assembly of any example herein, particularly example 108, wherein each bending point comprises a notch, and wherein the notches disposed along the first circumferential support section and the fourth circumferential support section are formed at an inner side of the plug.

Example 115. The assembly of any example herein, particularly any one of examples 63 to 93, wherein the plug comprises a plurality of struts that comprises a plurality of angled struts and a plurality of circumferential struts, wherein the plurality of angled struts are angled with respect to a central axis of the plug, and wherein the plurality of circumferential struts extend around a circumference of the plug.

Example 116. The assembly of any example herein, particularly example 115, wherein the plurality of struts intersect with each other at junctions, forming a plurality of cell openings therebetween.

Example 117. The assembly of any example herein, particularly example 116, wherein the plurality of cell openings comprises diamond-shaped cell openings, each of which is bound between four angled struts.

Example 118. The assembly of any example herein, particularly example 116 or 117, wherein the plurality of cell openings comprises triangular cell openings, each of which is bound between two angled struts and one circumferential strut.

Example 119. The assembly of any example herein, particularly any one of examples 115 to 118, wherein the plurality of circumferential struts comprises a rung of circumferential struts at the plug distal end, a rung of circumferential struts at the proximal end, and two intermediate rungs of circumferential struts at both ends of the intermediate portion.

Example 120. The assembly of any example herein, particularly any one of examples 63 to 119, wherein the introducer further comprises at least one stopper arm attached at a stopper proximal end to an outer surface of the inner shaft, and terminating with a stopper distal free end which is biased radially outward, wherein the stopper distal free end is axially positioned distal to the outer shaft proximal end when the plug is in the compacted configuration, and is axially positioned proximal to the outer shaft proximal end when the plug is in the expanded configuration.

Example 121. The assembly of any example herein, particularly example 120, wherein the stopper arm is retained in a folded configuration between the inner shaft and the outer shaft when the plug is in the compacted configuration.

Example 122. The assembly of any example herein, particularly example 120 or 121, wherein the stopper arm is biased radially outward such that the stopper distal free end abuts the outer shaft proximal end when the plug is in the expanded configuration.

Example 123. The assembly of any example herein, particularly example 122, wherein the stopper arm is configured to prevent distal movement of the inner shaft relative to the outer shaft, and/or proximal movement of the outer shaft relative to the inner shaft, when the plug is in the expanded configuration.

Example 124. The assembly of any example herein, particularly any one of examples 120 to 123, wherein the at least one stopper arm comprises a plurality of stopper arms.

Example 125. The assembly of any example herein, particularly any one of examples 63 to 124, wherein the outer shaft and the inner shaft are flexible.

Example 126. The assembly of any example herein, particularly any one of examples 63 to 125, wherein the sheath is flexible.

Example 127. The assembly of any example herein, particularly any one of examples 63 to 126, wherein proximal movement of the plug in the expanded configuration through the sheath lumen is configured to cause the sheath to locally expand in response to the outwardly directed radial force provided by the plug.

Example 128. The assembly of any example herein, particularly any one of examples 63 to 126, wherein the sheath includes at least one polymeric layer that includes a plurality of longitudinally-extending folds when the sheath is in the natural diameter, and wherein proximal movement of the plug in the expanded configuration through the sheath lumen is configured to cause the sheath to expand radially by at least partially unfolding the plurality of longitudinally-extending folds.

Example 129. The assembly of any example herein, particularly example 128, wherein the at least partial unfolding of the plurality of longitudinally-extending folds is configured to cause a decrease in a wall thickness of the sheath.

Example 130. The assembly of any example herein, particularly any one of examples 63 to 126, wherein the sheath includes at least one polymeric layer that includes a plurality of longitudinally-extending folds when the sheath is in the natural diameter, the plurality of longitudinally-extending folds creating a plurality of circumferentially spaced ridges and a plurality of circumferentially spaced valleys, and wherein proximal movement of the plug in the expanded configuration through the sheath lumen is configured to cause the sheath to expand radially by levelling out the ridges and the valleys.

Example 131. The assembly of any example herein, particularly any one of examples 63 to 130, wherein the sheath comprises at least one layer of self-contracting material.

Example 132. A method, comprising:
inserting an introducer device assembly comprising a sheath and an introducer into a patient's vasculature, wherein the sheath extends around the introducer and has a sheath distal end and a sheath lumen, and is configured to expand in a direction radially outward from a central axis of the sheath, and wherein the introducer comprises a nosecone, an inner shaft having an inner shaft distal end attached to the nosecone, an outer shaft disposed around the inner shaft and having an outer shaft distal end proximal to the inner shaft distal end, and a plug disposed around the inner shaft and having a plug distal end affixed to the nosecone, a plug proximal end affixed to the outer shaft distal end, and an intermediate portion disposed between the plug distal end and the plug proximal end;
approximating the plug distal end and the plug proximal end to each other to expand the plug from a plug first diameter in a compacted configuration of the plug, to a second plug diameter in an expanded configuration of the plug; and
axially sliding the plug relative to the sheath to expand at least a portion of the sheath in a direction radially outward from the sheath lumen;
wherein the intermediate portion exhibits a uniform diameter equal to the second plug diameter along an intermediate portion length thereof in the expanded configuration.

Example 133. The method of any example herein, particularly example 132, wherein the step of inserting an introducer device assembly into the patient's vasculature comprises inserting a guidewire into the patient's vasculature, and inserting the introducer device assembly over the guidewire.

Example 134. The method of any example herein, particularly example 132 or 133, wherein the step of approximating the plug distal end and the plug proximal end to each other comprises axially pulling the inner shaft relative to the outer shaft.

Example 135. The method of any example herein, particularly any one of examples 132 to 134, wherein the step of approximating the plug distal end and the plug proximal end to each other comprises axially pushing the outer shaft relative to the inner shaft.

Example 136. The method of any example herein, particularly any one of examples 132 to 135, wherein the step of sliding the plug comprises axially moving the entire introducer without axially moving the inner shaft and the outer shaft relative to each other.

Example 137. The method of any example herein, particularly any one of examples 132 to 136, wherein the sheath comprises a distal end portion terminating at the sheath distal and comprising a tapered tip portion configured to radially expand upon application of radially-oriented force thereto, and wherein inserting an introducer device assembly comprises advancing of the tapered tip portion through the patient's vasculature in a non-expanded state of the distal end portion of the sheath.

Example 138. The method of any example herein, particularly any one of examples 132 to 137, further comprising advancing the plug in its compacted configuration beyond the sheath distal end prior to expanding it, such that expansion of the plug is performed while the plug is distal to the sheath distal end.

Example 139. The method of any example herein, particularly example 137, further comprising positioning the plug in its compacted configuration proximal to the sheath distal end prior to expanding it, such that expansion of the plug is performed while the plug is at least partially disposed within the sheath.

Example 140. The method of any example herein, particularly example 139, wherein axially sliding the plug comprises distally pushing the plug through the distal end portion of the sheath, thereby expanding the distal end portion of the sheath.

Example 141. The method of any example herein, particularly example 138, wherein axially sliding the plug further comprises proximally pulling the plug in its expanded configuration through the sheath lumen.

Example 142. The method of any example herein, particularly example 141, wherein sliding the plug in a proximal direction comprises retracting the expanded plug back into the sheath, followed by sliding thereof through the sheath lumen.

Example 143. The method of any example herein, particularly any one of examples 132 to 142, wherein the step of inserting an introducer device assembly into the patient's vasculature comprises advancing the introducer device assembly while the nosecone is distal to the sheath distal end.

Example 144. The method of any example herein, particularly any one of examples 132 to 142, wherein the step of inserting an introducer device assembly into the patient's vasculature comprises advancing the introducer into the patient's vasculature, and then advancing the sheath over the introducer.

Example 145. The method of any example herein, particularly any one of examples 132 to 144, further comprising a step of withdrawing the introducer from the sheath lumen.

Example 146. The method of any example herein, particularly example 145, wherein withdrawing the introducer comprises moving the introducer axially towards a proximal end of the sheath until the introducer is completely removed from the sheath lumen.

Example 147. The method of any example herein, particularly example 145 or 146, further comprising advancing a prosthetic implant through the sheath lumen.

Example 148. The method of any example herein, particularly example 147, wherein advancing a prosthetic implant comprises advancing the prosthetic implant at a push force less than the push force required for advancement through a non-expanded sheath.

Example 149. The method of any example herein, particularly example 147, wherein the prosthetic implant is a prosthetic valve.

Example 150. The method of any example herein, particularly any one of examples 132 to 149, wherein the outer shaft defines an outer diameter, and wherein the intermediate portion length in the expanded configuration is greater than the outer diameter of the outer shaft.

Example 151. The method of any example herein, particularly example 150, wherein the intermediate portion length in the expanded configuration is greater than the outer diameter of the outer shaft by at least 20% of the outer diameter of the outer shaft.

Example 152. The method of any example herein, particularly example 151, wherein the intermediate portion length in the expanded configuration is greater than the outer diameter of the outer shaft by at least 30% of the outer diameter of the outer shaft.

Example 153. The method of any example herein, particularly example 151, wherein the intermediate portion length in the expanded configuration is greater than the outer diameter of the outer shaft by at least 50% of the outer diameter of the outer shaft.

Example 154. The method of any example herein, particularly example 151, wherein the intermediate portion length in the expanded configuration is greater than the outer diameter of the outer shaft by at least 75% of the outer diameter of the outer shaft.

Example 155. The method of any example herein, particularly any one of examples 132 to 154, wherein the intermediate portion length in the expanded configuration is at least as great as half the second plug diameter.

Example 156. The method of any example herein, particularly any one of examples 132 to 154, wherein the intermediate portion length in the expanded configuration is at least as great the second plug diameter.

Example 157. The method of any example herein, particularly any one of examples 132 to 154, wherein the intermediate portion length in the expanded configuration is at least 1.5 times greater than the second plug diameter.

Example 158. The method of any example herein, particularly any one of examples 132 to 154, wherein the intermediate portion length in the expanded configuration is at least 2 times greater than the second plug diameter.

Example 159. The method of any example herein, particularly any one of examples 132 to 154, wherein the intermediate portion length in the expanded configuration is at least 3 times greater than the second plug diameter.

Example 160. The method of any example herein, particularly any one of examples 132 to 159, wherein the plug distal end is affixed to a proximal end of the nosecone.

Example 161. The method of any example herein, particularly any one of examples 132 to 160, wherein the plug proximal end is affixed to the outer shaft distal end.

Example 162. The method of any example herein, particularly any one of examples 132 to 161, wherein the plug further comprises a distal portion that tapers distally in the expanded configuration from the second plug diameter at the intermediate portion to the plug first diameter at the plug distal end.

Example 163. The method of any example herein, particularly any one of examples 132 to 162, wherein the plug further comprises a proximal portion that tapers proximally in the expanded configuration from the second plug diameter at the intermediate portion to the plug first diameter at the plug proximal end.

Example 164. The method of any example herein, particularly example 162, wherein the plug further comprises a proximal portion that tapers proximally in the expanded configuration from the second plug diameter at the intermediate portion to the plug first diameter at the plug proximal end, wherein the distal portion defines a first draft angle in the expanded configuration of the plug, and wherein the proximal portion defines a second draft angle in the expanded configuration of the plug.

Example 165. The method of any example herein, particularly example 164, wherein the second draft angle is more acute than the first draft angle.

Example 166. The method of any example herein, particularly example 164, wherein the first draft angle is more acute than the second draft angle.

Example 167. The method of any example herein, particularly any one of examples 164 to 166, wherein the second draft angle is less than 45 degrees.

Example 168. The method of any example herein, particularly any one of examples 164 to 166, wherein the second draft angle is less than 30 degrees.

Example 169. The method of any example herein, particularly any one of examples 164 to 168, wherein the first draft angle is less than 45 degrees.

Example 170. The method of any example herein, particularly any one of examples 164 to 168, wherein the first draft angle is less than 30 degrees.

Example 171. The method of any example herein, particularly any one of examples 132 to 170, wherein the second plug diameter is greater than the first plug diameter by at least 20% of the first plug diameter.

Example 172. The method of any example herein, particularly any one of examples 132 to 170, wherein the second plug diameter is greater than the first plug diameter by at least 30% of the first plug diameter.

Example 173. The method of any example herein, particularly any one of examples 132 to 170, wherein the second plug diameter is greater than the first plug diameter by at least 50% of the first plug diameter.

Example 174. The method of any example herein, particularly any one of examples 132 to 170, wherein the second plug diameter is greater than the first plug diameter by at least 75% of the first plug diameter.

Example 175. The method of any example herein, particularly any one of examples 132 to 174, wherein the plug comprises a plurality of struts longitudinally extending between the plug distal end and the plug proximal end.

Example 176. The method of any example herein, particularly example 175, wherein the plurality of struts are circumferentially spaced from each other.

Example 177. The method of any example herein, particularly example 175 or 176, wherein each strut of the plurality of struts comprises a plurality of strut sections, separated from each other by bending point.

Example 178. The method of any example herein, particularly example 177, wherein the plurality of strut sections of each strut comprises a first strut section, a second strut section, and a third strut section.

Example 179. The method of any example herein, particularly example 178, wherein the intermediate portion comprises the second strut sections.

Example 180. The method of any example herein, particularly example 178 or 179, wherein the second strut sections remain parallel to a central axis of the plug both in the compacted configuration and the expanded configuration.

Example 181. The method of any example herein, particularly any one of examples 178 to 180, wherein each strut of the plurality of struts further comprises a first strut end portion extending from the plug distal end to a corresponding bending point separating it from the corresponding first strut section, and a second strut end portion extending from the plug proximal end to another corresponding bending point separating it from the corresponding third strut section.

Example 182. The method of any example herein, particularly example 181, wherein the first strut end portions and the second strut end portions remain unmoved during the transition the compacted configuration to the expanded configuration of the plug.

Example 183. The method of any example herein, particularly any one of examples 178 to 182, wherein each bending point comprises a notch.

Example 184. The method of any example herein, particularly example 183, wherein the notches disposed between each two adjacent strut sections are formed at an inner side of the struts.

Example 185. The method of any example herein, particularly example 181, wherein each bending point comprises a notch, and wherein the notches disposed between strut end portions and adjacent strut sections are formed at an outer side of the struts.

Example 186. The method of any example herein, particularly example 183, wherein expanding the plug comprises bending the first strut sections radially outward with respect to the first end portions, and bending the third strut sections radially outward with respect to the second end portions.

Example 187. The method of any example herein, particularly any one of examples 132 to 174, wherein the plug comprises a plurality of axial support sections and a plurality of circumferential support sections, wherein the plurality of axial support sections are parallel, in the compacted configuration, to a central axis of the plug, and wherein the plurality of circumferential support sections extend around a circumference of the plug.

Example 188. The method of any example herein, particularly example 187, wherein the plurality of axial support sections intersect with the plurality of circumferential support sections at bending points, forming a plurality of slots therebetween.

Example 189. The method of any example herein, particularly example 188, wherein the plurality of slots are rectangularly shaped.

Example 190. The method of any example herein, particularly any one of examples 187 to 189, wherein the plurality of circumferential support sections comprises a first circumferential support section at the plug distal end, a second circumferential support section proximal to the first circumferential support section, a third circumferential support section proximal to the second circumferential support section, and a fourth circumferential support section at the plug proximal end.

Example 191. The method of any example herein, particularly example 190, wherein the plurality of axial support sections comprises first axial support sections extending between the first circumferential support section and the second circumferential support section, second axial support sections extending between the second circumferential support section and the third circumferential support section, and third axial support sections extending between the third circumferential support section and the fourth circumferential support section.

Example 192. The method of any example herein, particularly example 191, wherein the intermediate portion comprises the second axial support sections.

Example 193. The method of any example herein, particularly example 191 or 192, wherein the second axial support sections remain parallel to the central axis of the plug both in the compacted configuration and the expanded configuration.

Example 194. The method of any example herein, particularly example 188, each bending point comprises a notch.

Example 195. The method of any example herein, particularly example 194, wherein the notches disposed between each two adjacent axial support sections are formed at an inner side of the plug.

Example 196. The method of any example herein, particularly example 190, wherein each bending point comprises a notch, and wherein the notches disposed along the first circumferential support section and the fourth circumferential support section are formed at an inner side of the plug.

Example 197. The method of any example herein, particularly any one of examples 132 to 174, wherein the plug comprises a plurality of struts that comprises a plurality of angled struts and a plurality of circumferential struts, wherein the plurality of angled struts are angled with respect to a central axis of the plug, and wherein the plurality of circumferential struts extend around a circumference of the plug.

Example 198. The method of any example herein, particularly example 197, wherein the plurality of struts intersect with each other at junctions, forming a plurality of cell openings therebetween.

Example 199. The method of any example herein, particularly example 198, wherein the plurality of cell openings comprises diamond-shaped cell openings, each of which is bound between four angled struts.

Example 200. The method of any example herein, particularly example 198 or 199, wherein the plurality of cell openings comprises triangular cell openings, each of which is bound between two angled struts and one circumferential strut.

Example 201. The method of any example herein, particularly any one of examples 197 to 200, wherein the plurality of circumferential struts comprises a rung of circumferential struts at the plug distal end, a rung of circumferential struts at the proximal end, and two intermediate rungs of circumferential struts at both ends of the intermediate portion.

Example 202. The method of any example herein, particularly any one of examples 132 to 201, wherein the introducer further comprises at least one stopper arm attached to an outer surface of the inner shaft, wherein the stopper arm is retained in a folded configuration between the inner shaft and the outer shaft when the plug is in the compacted configuration, and wherein the step of approximating the plug distal end and the plug proximal end to each other comprises exposing the stopper arm out of the outer shaft to allow them to spring radially away from the inner shaft.

Example 203. The method of any example herein, particularly example 202, wherein the stopper arm is configured to prevent distal movement of the inner shaft relative to the outer shaft, and/or proximal movement of the outer shaft relative to the inner shaft, when the plug is in the expanded configuration.

Example 204. The method of any example herein, particularly example 202 or 203, wherein the at least one stopper arm comprises a plurality of stopper arms.

Example 205. The method of any example herein, particularly any one of examples 132 to 204, wherein the outer shaft and the inner shaft are flexible.

Example 206. The method of any example herein, particularly any one of examples 132 to 205, wherein the sheath is flexible.

Example 207. The method of any example herein, particularly any one of examples 132 to 206, wherein the sheath includes at least one polymeric layer that includes a plurality of longitudinally-extending folds when the sheath is in a natural unexpanded diameter, and wherein the step of sliding the plug in a proximal direction comprises at least partially unfolding the plurality of longitudinally-extending folds.

Example 208. The method of any example herein, particularly example 207, wherein the step of at least partially unfolding the plurality of longitudinally-extending folds decreases a wall thickness of the sheath.

Example 209. The method of any example herein, particularly any one of examples 132 to 206, wherein the sheath includes at least one polymeric layer that includes a plurality of longitudinally-extending folds when the sheath is in a natural unexpanded diameter, the plurality of longitudinally-extending folds creating a plurality of circumferentially spaced ridges and a plurality of circumferentially spaced valleys, and wherein the step of sliding the plug in a proximal direction comprises levelling out the ridges and the valleys.

Example 210. The method of any example herein, particularly any one of examples 132 to 209, wherein the sheath comprises at least one layer of self-contracting material.

Example 211. An introducer, comprising:
a nosecone;
an inner shaft;
an outer shaft disposed around the inner shaft; and
a plug disposed around the inner shaft and positioned between the outer shaft and the nosecone;
wherein the plug is configured to expand from a plug first diameter in a compacted configuration of the plug, to a second plug diameter in an expanded configuration of the plug.

Example 212. The introducer of any example herein, particularly example 211, wherein the nosecone tapers from a nosecone proximal end to a nosecone distal tip.

Example 213. The introducer of any example herein, particularly example 211 or 212, wherein the inner shaft comprises an inner shaft distal end attached to the nosecone.

Example 214. The introducer of any example herein, particularly example 213, wherein the outer shaft comprises an outer shaft distal end and an outer shaft proximal end, wherein the outer shaft distal end is proximal to the inner shaft distal end.

Example 215. The introducer of any example herein, particularly any one of examples 211 to 214, wherein the plug comprises a plug distal end and a plug proximal end, wherein the plug distal end is affixed to the nosecone and the plug proximal end is affixed to the outer shaft distal end.

Example 216. The introducer of any example herein, particularly example 215, wherein the plug comprises a distal portion, a proximal portion, and an intermediate portion, wherein the distal portion extends proximally from the plug distal end, wherein the proximal portion extends distally from the plug proximal end, and wherein the intermediate portion extends between the distal portion and the proximal portion.

Example 217. The introducer of any example herein, particularly example 215 or 216, wherein approximation of the plug distal end and the plug proximal end is configured to cause the plug to expand from the plug first diameter to the plug second diameter.

Example 218. The introducer of any example herein, particularly example 217, wherein the intermediate portion has a uniform diameter equal to the second plug diameter along an intermediate portion length thereof when the plug is in the expanded configuration.

It is appreciated that certain features of the disclosed technology, which are, for clarity, described in the context of separate examples, may also be provided in combination in a single example. Conversely, various features of the disclosed technology, which are, for brevity, described in the context of a single example, may also be provided separately or in any suitable sub-combination or as suitable in any other described example of the disclosed technology. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. An introducer device assembly comprising an introducer, the introducer comprising:
a nosecone tapering from a nosecone proximal end to a nosecone distal tip;
an inner shaft having an inner shaft distal end attached to the nosecone;
an outer shaft disposed around the inner shaft, the outer shaft having an outer shaft distal end proximal to the inner shaft distal end, and an outer shaft proximal end; and
a plug disposed around the inner shaft, the plug having a plug distal end affixed to the nosecone, a plug proximal end affixed to the outer shaft, a distal portion extending proximally from the plug distal end, a proximal portion extending distally from the plug proximal end, and an intermediate portion between the distal portion and the proximal portion;
wherein the plug is configured to expand, upon approximation of the plug distal end and the plug proximal end to each other, from a plug first diameter in a compacted configuration of the plug, to a second plug diameter in an expanded configuration of the plug; and
wherein the intermediate portion exhibits a uniform diameter equal to the second plug diameter along an intermediate portion length thereof in the expanded configuration.

2. The assembly of claim 1, wherein the inner shaft and the outer shaft are axially movable relative to each other.

3. The assembly of claim 1, further comprising a sheath having a sheath distal end and a sheath lumen, wherein the sheath has a natural diameter in a non-expanded state thereof, and is expandable in a direction radially outward from a central axis of the sheath to a diameter which is greater than its natural diameter, and wherein the introducer is configured to move within the sheath lumen.

4. The assembly of claim 3, wherein proximal movement of the plug in the expanded configuration through the sheath lumen is configured to cause the sheath to locally expand in response to the outwardly directed radial force provided by the plug.

5. The assembly of claim 3, wherein the sheath includes at least one polymeric layer that includes a plurality of longitudinally-extending folds when the sheath is in the natural diameter, the plurality of longitudinally-extending folds creating a plurality of circumferentially spaced ridges and a plurality of circumferentially spaced valleys, and wherein proximal movement of the plug in the expanded configuration through the sheath lumen is configured to cause the sheath to expand radially by levelling out the ridges and the valleys.

6. The assembly of claim 1, wherein the intermediate portion length in the expanded configuration is at least as great as half the second plug diameter.

7. The assembly of claim 1, wherein the outer shaft defines an outer diameter, and wherein the intermediate portion length in the expanded configuration is greater than the outer diameter of the outer shaft.

8. The assembly of claim 1, wherein the plug comprises a plurality of struts longitudinally extending between the plug distal end and the plug proximal end.

9. The assembly of claim 1, wherein the plug comprises a plurality of axial support sections and a plurality of circumferential support sections, wherein the plurality of axial support sections are parallel, in the compacted configuration, to a central axis of the plug, and wherein the plurality of circumferential support sections extend around a circumference of the plug.

10. The assembly of claim 1, wherein the plug comprises a plurality of struts that comprises a plurality of angled struts and a plurality of circumferential struts, wherein the plurality of angled struts are angled with respect to a central axis of the plug, and wherein the plurality of circumferential struts extend around a circumference of the plug.

11. The assembly of claim 1, wherein the introducer further comprises at least one stopper arm attached at a stopper proximal end to an outer surface of the inner shaft, and terminating with a stopper distal free end which is biased radially outward, wherein the stopper distal free end is axially positioned distal to the outer shaft proximal end when the plug is in the compacted configuration, and is axially positioned proximal to the outer shaft proximal end when the plug is in the expanded configuration.

12. The assembly of claim 11, wherein the stopper arm is retained in a folded configuration between the inner shaft and the outer shaft when the plug is in the compacted configuration.

13. The assembly of claim 11, wherein the stopper arm is biased radially outward such that the stopper distal free end abuts the outer shaft proximal end when the plug is in the expanded configuration.

14. The assembly of claim 13, wherein the stopper arm is configured to prevent distal movement of the inner shaft relative to the outer shaft, and/or proximal movement of the outer shaft relative to the inner shaft, when the plug is in the expanded configuration.

15. A method comprising:
inserting an introducer device assembly comprising a sheath and an introducer into a patient's vasculature, wherein the sheath extends around the introducer and has a sheath distal end and a sheath lumen, and is configured to expand in a direction radially outward from a central axis of the sheath, and wherein the introducer comprises a nosecone, an inner shaft having an inner shaft distal end attached to the nosecone, an outer shaft disposed around the inner shaft and having an outer shaft distal end proximal to the inner shaft distal end, and a plug disposed around the inner shaft and having a plug distal end affixed to the nosecone, a plug proximal end affixed to the outer shaft distal end, and an intermediate portion disposed between the plug distal end and the plug proximal end;
approximating the plug distal end and the plug proximal end to each other to expand the plug from a plug first diameter in a compacted configuration of the plug, to a second plug diameter in an expanded configuration of the plug; and
axially sliding the plug relative to the sheath to expand at least a portion of the sheath in a direction radially outward from the sheath lumen;
wherein the intermediate portion exhibits a uniform diameter equal to the second plug diameter along an intermediate portion length thereof in the expanded configuration.

16. The method of claim 15, wherein the outer shaft defines an outer diameter, and wherein the intermediate portion length in the expanded configuration is greater than the outer diameter of the outer shaft.

17. The method of claim 15, wherein the intermediate portion length in the expanded configuration is at least as great as half the second plug diameter.

18. The method of claim 15, wherein the sheath includes at least one polymeric layer that includes a plurality of longitudinally-extending folds when the sheath is in a natural unexpanded diameter, and wherein the step of sliding the plug in a proximal direction comprises at least partially unfolding the plurality of longitudinally-extending folds.

19. The method of claim 18, wherein the step of at least partially unfolding the plurality of longitudinally-extending folds decreases a wall thickness of the sheath.

20. The method of claim 15, wherein the sheath includes at least one polymeric layer that includes a plurality of longitudinally-extending folds when the sheath is in a natural unexpanded diameter, the plurality of longitudinally-extending folds creating a plurality of circumferentially spaced ridges and a plurality of circumferentially spaced valleys, and wherein the step of sliding the plug in a proximal direction comprises levelling out the ridges and the valleys.
